(19) United States
(12) Patent Application Publication
(10) Pub. No.: US 2023/0314426 A1
(43) Pub. Date: Oct. 5, 2023

(54) ANTI-ARGONAUTE PROTEIN AUTOANTIBODIES AS BIOMARKERS OF AUTOIMMUNE NEUROLOGICAL DISEASES

(71) Applicants: UNIVERSITE CLAUDE BERNARD LYON 1, VILLEURBANNE (FR); CENTRE NATIONAL DE LA RECHERCHE SCIENTIFIQUE (CNRS), PARIS (FR); HOSPICES CIVILS DE LYON, LYON (FR); UNIVERSITE JEAN MONNET SAINT ETIENNE, SAINT ETIENNE (FR); CENTRE HOSPITALIER UNIVERSITAIRE DE SAINT ETIENNE, SAINT ETIENNE (FR); INSERM (Institut National de la Sante et de la Recherche Medicale), PARIS (FR)

(72) Inventors: Le Duy DO, LYON (FR); Jerome HONNORAT, BRON (FR); Anne-Laurie PINTO, VILLEURBANNE (FR); Christian Peter MORITZ, SAINT ETIENNE (FR); Jean-Christophe ANTOINE, CHALAIN-LE-COMTAL (FR); Jean-Philippe CAMDESSANCHE, SAINT ETIENNE (FR); Sergio MUNIZ-CASTRILLO, LYON (FR)

(21) Appl. No.: 18/044,008

(22) PCT Filed: Sep. 14, 2021
(86) PCT No.: PCT/EP2021/075239

§ 371 Date: Mar. 3, 2023

(30) Foreign Application Priority Data
Sep. 15, 2020 (EP) 20306030.6

(51) Int. Cl. G01N 33/564

(52) U.S. Cl. CPC G01N 33/564; G01N 2800/28; G01N 2800/52

(57) ABSTRACT

The invention relates to a process for identifying patients affected by an autoimmune neurological disease, comprising a step of detection of at least one type of anti-Argonaute autoantibodies (AGO-Abs) in a biological sample of an individual susceptible to be affected by said disease, wherein positive detection of said at least one type of AGO-Abs means that said individual is affected by said autoimmune neurological disease.

### FIELD OF THE INVENTION

The present invention relates to processes of diagnosis of human diseases.

In particular, the present invention relates to a process for identifying patients affected by an autoimmune neurological disease. The present invention also relates to a process of classification of patients, comprising identifying among a group of patients affected with neurological signs and symptoms, a subgroup of patients affected with an autoimmune neurological disease. The present invention also concerns a specific biomarker of the autoimmune nature of neurological diseases.

### BACKGROUND OF THE INVENTION

Paraneoplastic neurological syndromes (PNS), autoimmune encephalitis, and inflammatory neuropathies are rare diseases in which an autoimmune response against the nervous system is responsible for the neurological disorder; this autoimmune response is triggered by cancer in PNS. All these pathologies are characterized by the presence, in serum and/or cerebrospinal fluid (CSF) of affected patients, of autoantibodies that recognize neural antigens. These autoantibodies can be used as biomarkers, and they make it possible to assert the autoimmune nature of the pathology.

The identification of biomarkers is essential since the clinical presentation of these diseases is non-specific, and many other etiologies may cause the same signs and symptoms. Thus, among neurological disorders, identification of those with an autoimmune origin is essential for diagnosis and then appropriate therapeutic strategies.

The diagnosis of autoimmune origin is currently based on the detection of disease-specific autoantibodies in serum and/or CSF. To date, about twenty autoantibodies have been described for various autoimmune neurological diseases (Graus et al., 2004; Dalmau et al., 2017; Querol et al., 2017). For example, antibodies against fibroblast growth factor receptor 3 (FGFR-3) are specifically identified in a subgroup of patients with sensory neuronopathy of autoimmune origin (Antoine et al., 2015). Diagnostically useful autoantibodies and related assays are also disclosed in U.S. Pat. Nos. 10,539,577, 9,766,253, 10,539,577, 7,314,721, EP 2952898, EP 3086120 and patent applications US 2017/0343564, EP 3026434 and EP 3101424.

Specificities of these autoantibodies are very strong. The description of these autoantibodies over the last 30 years has transformed patient management and has substantially increased the number of patients identified each year.

However, there are still a large number of patients with a clinical presentation compatible with an autoimmune origin, and in whom no classical autoantibodies have been identified. In these cases, it is always difficult to establish the autoimmune nature of their condition, which is only sometimes suggested by inflammatory abnormalities in the CSF, the disease course, or the co-occurrence of other autoimmune diseases. In many cases, there is no indirect clues suggesting an autoimmune origin, and the characterization of circulating autoantibodies would be the only way to identify the autoimmune origin of the disease.

Hence, the discovery of new autoantibodies serving as biomarkers is of major importance for the assertion of the autoimmune origin of these neurological disorders, in order to propose an adapted immunomodulatory treatment to the affected patients.

Autoantibodies Against Argonaute Protein Family (AGO-Abs)

Argonaute (AGO) proteins constitute a highly conserved family of RNA-binding proteins. They were named from an “AGO”-knockout phenotype of Arabidopsis thaliana. AGO proteins are involved in complexes comprising small RNAs, and they are guided to complementary sites on target RNA molecules, where they play a key role in the mechanism of RNA silencing, by repressing translation through the interaction with microRNAs and short interfering RNAs (Meister et al., 2005; Peters Et Meister, 2007).

This protein family comprises four RNA-binding proteins named AGO1, AGO2, AGO3 and AGO4.

Initially, AGO-Abs were labeled as Su-Abs, based on the initials of the first patient from whom these antibodies were isolated, since the antigen was still unknown. Later, AGO-Abs were reported in the serum of patients with systemic lupus erythematosus, scleroderma, Sjögren syndrome, and other autoimmune rheumatologic diseases (Satoh et al., 1994; Satoh et al., 2013). The “Su” antigen was identified by immunoprecipitation as the 100 kDa RNA-binding protein AGO2. It is localized in the cytoplasmic structures designed as GW/P-bodies, involved in mRNA processing and RNA interference. These cytoplasmic structures also include other components such as the TNRC6 (trinucleotide repeat containing) proteins.

In a study investigating the clinical correlation of antibodies against several antigens present in the GW/P-bodies, two out of six patients with serum AGO2-Abs had peripheral, sensory-motor neuropathies, and one patient with Sjögren syndrome had a non-specified ataxia (Bhanji et al., 2007). This study found antibodies against AGO2 but none directed to the other AGO proteins. Furthermore, this study was based only on serum analysis, and no precise clinical descriptions of patients were provided.

The problem underlying the present invention is the implementation of a diagnosis process allowing to distinguish patients affected by an autoimmune neurological disease, from patients affected by a neurological disease of non-autoimmune origin.

The inventors have identified autoantibodies against Argonaute protein family (AGO-Abs) in patients with suspected autoimmune neurological disorders, and they have established the correlation between the presence of at least one AGO-Ab and the autoimmune nature of a neurological disorder. Based on these results, it is hereby proposed that AGO-Abs are useful biomarkers for autoimmunity, allowing the definition of a subgroup of patients affected with autoimmune neurological diseases.

### SUMMARY OF THE INVENTION

In a first embodiment, the present invention concerns a process for identifying patients affected by an autoimmune neurological disease, comprising a step of detection of at least one type of anti-Argonaute autoantibodies (AGO-Abs) in a biological sample of an individual susceptible to be affected by said disease, wherein positive detection of said at least one type of AGO-Abs means that said individual is affected by said autoimmune neurological disease.

In other words, the problem underlying the present invention is solved by a method comprising a step of detecting the presence or absence of an autoantibody to an AGO protein, preferably selected from the group comprising an autoantibody to AGO1, AGO2, AGO3 and AGO4. Preferably detection of one or more of these autoantibodies in a sample from a patient indicates an increased likelihood that the tested patient suffers or will likely suffer from an autoimmune neurological disease. In another embodiment, detection of an elevated level of one or more of these autoantibodies in a sample from a patient, compared to the average level observed in a sample of an healthy subject, indicates an increased likelihood that the tested patient suffers from an autoimmune neurological disease.

In particular, said identified patients are affected by an autoimmune neurological disease chosen among the group consisting of: autoimmune encephalitis, paraneoplastic neurological syndromes (PNS), and inflammatory peripheral neuropathies; and particular clinical phenotypes are chosen among the group consisting of: sensory neuronopathy (SNN), limbic encephalitis, cerebellar syndrome, other inflammatory peripheral neuropathies such as small fiber neuropathy, chronic inflammatory demyelinating polyneuropathy, rhombencephalitis, and opsoclonus-myoclonus.

In a second embodiment, the present invention concerns a process of classification of patients, comprising:
a. identifying among a group of patients affected with neurological diseases a subgroup of patients affected with an autoimmune neurological disease, wherein the step of identification is made by the process described above, and
b. classifying said patients.

In a third embodiment, the present invention concerns a biomarker specific of an autoimmune nature of a neurological disease, consisting of AGO-Abs, in particular directed against at least one of the following proteins: AGO1, AGO2, AGO3, AGO4, and combinations thereof.

In other words, the problem is solved by an autoantibody from the group comprising an autoantibody to AGO1, an autoantibody to AGO2, an autoantibody to AGO3 and an autoantibody to AGO4. Preferably the autoantibody is directed against AGO1, AGO3 and/or AGO4. Preferably the autoantibody is purified, isolated, diluted and/or enriched.

In a fourth embodiment, the present invention concerns a kit for the implementation of processes as defined above, comprising means for the detection and/or quantification of at least one type of AGO-Abs in a biological sample of an individual.

In other words, the problem is solved by a kit comprising a means for the detection and/or quantification of an autoantibody to an AGO, preferably from the group comprising an autoantibody to AGO1, an autoantibody from the group comprising an autoantibody to AGO2, an autoantibody from the group comprising an autoantibody to AGO3 and an autoantibody from the group comprising an autoantibody to AGO4. The means is preferably selected from the group comprising a secondary antibody, preferably to a human antibody, more preferably to a human IgG class antibody, and a polypeptide comprising an AGO or a variant thereof, preferably selected from the group comprising AGO1, AGO2, AGO3 and AGO4, which is preferably labelled with a detectable label. Any kit may comprise a positive control, for example a recombinant antibody to an AGO4, preferably from the group comprising AGO1, AGO2, AGO3 and AGO4, and a negative control, for example a sample from a healthy subject. The kit may comprise a set of calibrators.

In a fifth embodiment, the present invention concerns a process of treatment of an autoimmune neurological disease in patients in need thereof, comprising the following steps:
a) classifying patients affected with neurological diseases according to the process of classification as defined above, and
b) administering an appropriate immunomodulatory compound to a subgroup of patients identified as being affected with an autoimmune neurological disease.

### BRIEF DESCRIPTION OF THE FIGURES

FIG. 1: Immunostaining of adult rat brain with CSF (1:10) of patient XI. Incubation with the patient's CSF resulted in strong reactivity with the stratum pyramidal of the hippocampus, granules cells of the cerebellum, and cerebral cortex. Scale bar is indicated for each magnification.

FIG. 2. Immunoprecipitation of the antigens with patient XI's CSF. Note the specifically enriched band around 100 kDa (*) and the less intense band around 260 kDa (**) obtained after immunoprecipitation with patient XI's CSF (E+) compared to control CSF (E−). Protein bands were visualized via silver staining.

FIG. 3.

A. Protein array analysis. Visualized reactivities of the tested patients with sensory neuronopathy (SN), other peripheral neuropathies (ONP), and healthy controls (HC) with AGO1. Four SN patients were significantly positive (>4 SDs above samples from patients with other neuropathies and healthy controls; with grey background).

B. Quantification of the normalized reactivities of the tested patient sera with seven AGO variants. Unfilled circles represent significantly positive samples. Norm. signal int.=normalized signal intensities; a.u.=arbitrary units, AGO2_frag: fragment of AGO2.

FIG. 4. AGO2 reactivities of all ELISA-AGO1-Abs-positive sera. ELISA reactivities of all ELISA-AGO1-Abs-positive sera with AGO2 shown as a correlation plot of AGO2 DOD versus AGO1 ΔOD. Black points: AGO2-positive; grey points: AGO2-negative (defined with a cut-off of z=4 above HC; horizontal line). Dashed line distinguishes between strong (z>14 above HC; “++”) and intermediate (z>4, <14 above HC; “+”) AGO1-Abs-positive patients. Reactivities are plotted as arithmetic differences of the optical density of AGO1-coated versus non-coated ELISA wells (ΔOD).

FIG. 5. Patient screening via ELISA: Reactivities of 606 human sera with AGO1. Significantly positive reactivities (z>4 above HC; solid line) were found in 26/278 peripheral neuropathies (9%; in detail: 19/131 sensory neuronopathy (SNN; 15%), 2/67 small-fiber neuropathy (SFN; 3%), 5/80 other peripheral neuropathies (OPN; 6%), 5/74 central nervous system diseases (CNSD; 7%), 9/127 autoimmune diseases (AID; 7%), and 0/116 healthy controls (HC; 0%). Dashed line distinguishes between strong (z>14 above HC; “++”) and intermediate (z>4, <14 above HC; “+”) AGO1-Abs-positive patients. Reactivities are plotted as arithmetic differences of the optical density of AGO1-coated versus non-coated ELISA wells (ΔOD).

FIG. 6. Establishment of a sensitive ELISA approach to detect and distinguish conformation-specific AGO1 Abs.

A. ELISA establishment using 16 out of the 21 AGO Abs CBA-positive neurological patients, 3 AGO Abs CBA-positive control patients, 10 healthy controls, and 1 commercial anti-AGO1 antibody. The method establishment included three conditions of AGO1 protein: standard conditions (using a common coating buffer), stabilizing conditions (30% glycerol in coating buffer), and linearizing conditions (i.e., denaturizing conditions using 0.8% SDS). For each of the three conditions a respective positivity threshold was defined based on the ODs of the 10 healthy controls under the respective conditions (horizontal bars=arithmetic mean plus 3 standard deviations).

B. Same as A, but showing 27 novel AGO Abs-positive patients detected by using a large screening of 823 sera (including 116 healthy controls that were used to define positivity, threshold at 4 standard deviations above their mean) using the established ELISA approach under stabilizing conditions. Black curves: patients with conformation-specific reactivity pattern; grey curves: patients with non-conformation-specific reactivity pattern.

FIG. 7. Frequencies and characteristics of AGO1 Abs in different disease groups.

A. ELISA ΔODs plotted for different disease groups comprising 813 subjects.

B. Correlation analysis between AGO1 Abs titers and ELISA ΔODs.

C. Correlation analysis between ELISA ΔODs for AGO2 Abs and AGO1 Abs.

D. Correlation analysis between ELISA z-scores for AGO2 Abs and AGO1 Abs.

FIG. 8. ELISA reactivities with study cohorts as well as frequencies and titers of AGO Abs

SNN: sensory neuronopathy; SFN: small-fiber neuronopathy; CIDP: chronic inflammatory demyelinating polyneuropathies; ONP: other peripheral neuropathies; SjS: Sjögren Syndrome; HC: Healthy controls; AID: autoimmune diseases; ΔOD: Difference of optical density between AGO1-coated versus non-coated ELISA wells.

A. Serum reactivities (shown as ΔODs) with AGO1 of 823 subjects among 8 study groups via ELISA. The solid line at ΔOD=0.386 represents the z=4 cutoff line. The dashed line at ΔOD=1.21 (z=14) distinguishes between moderately positive (z=4-14, +) and strongly positive (z>14, ++) subjects. Absolute and relative numbers of seropositive cases are shown in the table below the graph.

B. Frequency of AGO1 Abs among different study groups depending on the Ab level (left: moderately positive [+], right: strongly positive [+]).

FIG. 9. AGO Abs frequency in different subgroups with and without dysimmune context

A-C. Frequencies of AGO1 Abs among the study groups depending on the general dysimmune status

A. comparing frequencies in study groups with versus without a dysimmune context;

B. comparing frequencies among different study groups without any dysimmune context;

C. comparing frequencies among different study groups with dysimmune context.

D-F. Frequencies of AGO1 Abs among the study groups depending on the diagnosed autoimmune status

D. comparing frequencies in study groups with versus without an accompanying autoimmune disease;

E. comparing frequencies among different study groups without an accompanying autoimmune disease;

F. comparing frequencies among different study groups with an accompanying autoimmune disease.

*p≤0.05, **p≤0.01, ***p≤0.001, ns=not significant, p>0.05, Fisher's exact test.

### DETAILED DESCRIPTION OF EMBODIMENTS OF THE INVENTION

The present invention relates to a process for identifying patients affected by an autoimmune neurological disease, comprising a step of detection of at least one type of anti-Argonaute autoantibody (AGO-Ab) in a biological sample of an individual susceptible to be affected by said disease, wherein positive detection of said at least one type of AGO-Abs means that said individual is affected by said autoimmune neurological disease.

Unless stated otherwise, the following terms and phrases as used herein are intended to have the following meanings:

A “neurological disease” refers to a pathologic disorder of the nervous system, i.e. in the brain, spinal cord, or nerves of the human body.

Common signs and symptoms observed in neurological diseases are for example: altered level of consciousness, confusion, memory impairment, seizures, behavioural disturbances, sleep disorders, slurred speech, swallowing difficulty, altered ocular movements, gait instability, poor coordination, dizziness, paralysis and weakness, loss of sensation, paraesthesia, dysesthesia, pain.

These signs and symptoms are assessed and established by neurological examination, by a health provider or clinician.

An “autoimmune neurological disease” refers to neurological diseases caused by an abnormal immune reaction directed against the own cells of the body, which is in particular suggested by the presence of autoantibodies.

In a preferred embodiment, the autoimmune neurological disease can be diagnosed using the method or reagents according to the present invention, or they can aid in such a diagnosis.

In the sense of the invention, the terms “diagnosis” and “diagnostic”, herein used indifferently, are to be used in their broadest possible sense and may refer to any kind of procedure aiming to obtain information instrumental in the assessment whether a patient, known or an anonymous subject from a cohort, suffers or is likely or more likely than the average or a comparative subject, the latter preferably having similar symptoms, to suffer from a certain disease or disorder in the past, at the time of the diagnosis or in the future, to find out how the disease is progressing or is likely to progress in the future or to evaluate the responsiveness of a patient or patients in general with regard to a certain treatment, for example the administration of immunosuppressive drugs, or to find out whether a sample is from such a patient. Such information may be used for a clinical diagnosis, but may also be obtained by an experimental and/or research laboratory for the purpose of general research, for example to determine the proportion of subjects suffering from the disease in a patient cohort or in a population. In other words, the term “diagnosis” comprises not only diagnosing, but also prognosticating and/or monitoring the course of a disease or disorder, including monitoring the response of one or more patients to the administration of a drug or candidate drug, for example to determine its efficacy.

In particular, the detection of an autoantibody to AGO1 indicates an increased likelihood that a patient suffers from or will suffer from SNN associated with a more severe and widespread disorder that may be improved by immunomodulatory treatments. Moreover, the titre of an autoantibody may be determined and monitored.

An increase in the autoantibody level may indicate an increased likelihood of a further progression or relapse of the disease and/or an unsuccessful therapy, while a decrease may indicate an increased likelihood of a remission and/or successful therapy. Preferably, the treatment is adjusted accordingly, for example by increasing the strength of or continuing an immunomodulatory treatment in the case of increased or increasing autoantibody titres or by decreasing or stopping the treatment in the case of decreased autoantibody titres, preferably if the autoantibodies can no longer be detected.

In a preferred embodiment, it is possible to distinguish a patient who is likely to respond positively or who is amenable to immunosuppressive treatment from one who is not.

While the result may be assigned to a specific patient for clinical diagnostic applications and may be communicated to a medical doctor or institution treating said patient, this is not necessarily the case for other applications, for example in diagnostics for research purposes, where it may be sufficient to assign the results to a sample from an anonymized patient. In another preferred embodiment, the detection of an autoantibody binding specifically to an AGO protein is considered to imply a definitive diagnosis of an autoimmune neurological disease, solely based on the presence of said autoantibody.

In the sense of the invention, an autoantibody designates an antibody targeting self-antigens, i.e., an antibody directed against one or more of the individual's own proteins.

In the present application, the terms “anti-AGO antibody” and “anti-AGO autoantibody” are used interchangeably; indeed, it is understood that in the sense of the invention, anti-AGO antibodies are autoantibodies, since AGO1, AGO2, AGO3, and AGO4 are autoantigens.

In the sense of the invention, the term “AGO”, as used herein, refers to any isoform of the human proteins selected from the group comprising AGO1, AGO2, AGO3 and AGO4. AGO protein sequences are available in public databases under the following references in the UNIPROT database:
AGO1 is referenced Q9UL18; the nucleotide sequence of the coding sequence is shown in SEQ ID NO. 5.
AGO2 is referenced Q9UKV8; the nucleotide sequence of the coding sequence is shown in SEQ ID NO. 6.
AGO3 is referenced Q9H9G7; the nucleotide sequence of the coding sequence is shown in SEQ ID NO. 7.
AGO4 is referenced Q9HCK5; the nucleotide sequence of the coding sequence is shown in SEQ ID NO. 8.

In the sense of the invention, the phrase “detection of at least one antibody” means a step of search, by any technique known by the person skilled in the art, in a biological sample of the individual to be tested, of at least one antibody.

The phrase “at least one antibody” is synonymous of “one or more antibodies”.

In the sense of the invention, the expressions “Argonaute autoantibodies”, “AGO-Abs” as well as “anti-Argonaute autoantibodies” all refer to autoantibodies directed against any member of the AGO protein family, and in particular antibodies against AGO1 and/or AGO2 and/or AGO3 and/or AGO4 and/or any fragment of any of these proteins.

In the sense of the invention, the phrase “detection of at least one type of Argonaute autoantibodies (AGO-Abs)” designates the detection of autoantibodies directed against any member of the AGO protein family, and in particular antibodies against AGO1 and/or AGO2 and/or AGO3 and/or AGO4, and/or any fragment of any of these proteins.

As reported herein, AGO-Abs commonly react against the four AGO proteins; this shared reactivity is linked to the high homology (80%) among the four AGO proteins (Jakymiw et al., 2006). Nevertheless, in the sense of the invention, AGO-Abs include AGO-Abs reacting specifically with only one AGO protein, AGO-Abs reacting with AGO1 and AGO2, AGO-Abs reacting with any combination of two AGO proteins (for example, reacting with AGO1 and AGO3), AGO-Abs reacting with any combination of three AGO proteins (for example, reacting with AGO1, AGO3 and AGO4), and AGO-Abs reacting with the four AGO proteins.

In the sense of the invention, “biological sample” designates any biological fluid obtained from a patient by any technique known by the person skilled in the art. In particular, the biological sample used in the process according to the invention is chosen among the following group: cerebrospinal fluid, serum, plasma, whole blood, urine, lymph, saliva, sputum, seminal fluid, and tears. The sample is preferably plasma, whole blood, serum or cerebrospinal fluid.

In a preferred embodiment, the biological sample is cerebrospinal fluid.

In another preferred embodiment, the biological sample is serum.

In a specific embodiment of the invention, the step of detection of AGO-Abs is performed by cell-based assay (CBA) with a panel of cells, each cell expressing separately AGO1, AGO2, AGO3, or AGO4 protein, or at least one of their fragments.

In the sense of the present invention, a CBA corresponds to an assay wherein cells expressing at least one AGO protein are incubated with a biological sample of an individual, in order to identify if said biological sample comprises at least one type of AGO-Abs.

The biological sample may have been pre-treated, for example diluted, before performing the CBA.

In a specific embodiment, cells expressing an AGO protein have been transformed or genetically modified to express an exogenous AGO protein. In this case, these cells overexpress the exogenous nucleic acid molecule encoding AGO protein.

Cells of the CBA may express one or more AGO proteins. Preferentially, cells express a single AGO protein selected among AGO1, AGO2, AGO3 and AGO4. More preferentially, the CBA uses a panel of cells, comprising at least one of the following:
cells expressing AGO1 or a variant thereof;
cells expressing AGO2 or a variant thereof;
cells expressing AGO3 or a variant thereof;
cells expressing AGO4 or a variant thereof; and
cells expressing any fragment of AGO1, AGO2, AGO3, or AGO4.

In the sense of the invention, the term “fragment” designates an antigenic fragment, which is a portion of the whole AGO protein that contains at least one epitope allowing its binding with at least one anti-AGO antibody.

In the sense of the invention, the term “variant thereof” refers to a polypeptide comprising amino acid sequences that are at least 40, 50, 60, 70, 75, 80, 85, 90, 92, 94, 95, 96, 97, 98, 99, 99.3, 99.4, 99.5, 99.6, 99.7, 99.8 or 99.9% identical to the reference amino acid sequence of the AGO protein chosen from the group comprising AGO1, AGO2, AGO3 and AGO4, wherein amino acids other than those essential for the biological activity, for example the ability of an antigen to bind to an (auto)antibody, or the fold or structure of the polypeptide are deleted or substituted and/or one or more such essential amino acids are replaced in a conservative manner and/or amino acids are added such that the biological activity of the polypeptide is preserved.

The variant contains at least one epitope allowing its binding with at least one anti-AGO antibody.

The variant may also be a fragment of an AGO protein, preferably a fragment consisting of a number of consecutive amino acids from the whole AGO protein, which number covers at least 10, 20, 30, 40, 50, 60, 70, 80, 90, 95, 96, 97, 98, 99% of the unaltered sequence of the AGO protein chosen from the group comprising AGO1, AGO2, AGO3 and AGO4. The fragment may be fused N-terminally or C-terminally with other known polypeptides or artificial sequences such as linkers, and comprise active portions or domains, for example linkers, affinity tags or purification tags. The variant may also comprise another autoantigen or a variant thereof.

The variant may also comprise one or more copies of one or more AGO protein (s) different than the AGO protein at the origin of the variant.

In particular, the variant may comprise a folded fragment comprising at least 10, 20, 30 or 40 successive amino acids of the sequence of the AGO protein.

In a preferred embodiment, the panel of cells comprises these four types of cells, each cell expressing separately AGO1, AGO2, AGO3, or AGO4 protein, or at least one of their fragments.

In a specific embodiment, the panel of cells comprises cells of the same origin, for example issued from a same line of immortalized cells.

The person skilled in the art knows well the conditions for performing a CBA and will be able to adapt the buffer composition, time of incubation, number of cells, concentration of the biological sample, and use of detection reagents, according to the general knowledge.

In another specific embodiment of the invention, the step of detection of AGO-Abs is performed by an immunoassay. An immunoassay corresponds to any assay that detects the presence of a molecule in a solution through the use of an antibody or an antigen, specific for said molecule to be detected.

Such immunoassay is in particular chosen among the group consisting of:
ELISA (enzyme-linked immunosorbent assay),
immunoblot, for example dot blot, western blot, line assay,
radio-immunoassay,
chemiluminescent immunoassay,
electro-chemiluminescence immunoassay, and
immunofluorescence assay.

In a specific embodiment, the step of detection of AGO-Abs is performed by an ELISA assay using wells coated with a recombinant AGO protein or fragment issued from said recombinant protein, in particular with at least one recombinant protein or fragment chosen among: AGO1, AGO2, AGO3, AGO4, and a fragment thereof.

Such ELISA assay may be performed under different conditions, in particular:
using a standard coating buffer, or
using a conformation-stabilizing coating buffer (for example, with 30% of glycerol in a standard coating buffer), or
using a denaturizing coating buffer, adapted for linearizing proteins (for example, with 0.8% SDS in a standard coating buffer).

In a preferred embodiment, the ELISA assay is performed under conformation-stabilizing conditions.

As it is known by the person skilled in the art, to improve the sensitivity and/or specificity of the ELISA test, the serum-specific background noise may be determined and subtracted (cf. ΔOD) for each tested sample.

Other techniques may be used for the detection of AGO-Abs. In particular, in the examples of the present application, the following techniques are described:
immunoprecipitation with or without subsequent mass spectrometry, and
protein micro-arrays.

Although the last two techniques are less easily adaptable for an industrial application, they are nevertheless included into the scope of the invention as well.

In a specific embodiment, the AGO-Abs are detected using a method selected from the group comprising: immunodiffusion, immunoelectrophoresis, light scattering immunoassays, bead-based immunoassays, agglutination, labeled immunoassays such as those from the group comprising radiolabeled immunoassay, enzyme immunoassays, more preferably ELISA, chemiluminescence immunoassays, preferably electrochemiluminescence immunoassay, and immunofluorescence, preferably indirect immunofluorescence.

Advantageously, the step of detection of at least one type of AGO-Abs is coupled with a step of quantification of said autoantibodies present in the biological sample of the tested individual. This quantification step can be performed by any technique known by the person skilled in the art. In particular, the performed CBA or immunoassay may use a serial dilution technique, and/or a calibrator or internal control for quantifying AGO-Abs. Table 3 in the examples section shows titres of AGO-Abs found in CSF and sera of tested patients.

Concerned Diseases and Patients Susceptible to be Affected by Said Diseases

In the sense of the invention, “an individual susceptible to be affected by an autoimmune neurological disease” refers to any individual presenting at least one neurological sign and/or symptom such as listed previously.

This individual may be affected by a neurological disease as established after clinical examination by a physician and/or by biological analysis.

In a specific embodiment, this individual susceptible to be affected by an autoimmune neurological disease presents with neurological signs and/or symptoms, in particular is affected by one of the following syndromes: limbic encephalitis, cerebellar syndrome, and/or sensory neuronopathy.

In a specific embodiment, the process of the invention allows to diagnose one of the autoimmune neurological diseases chosen among the group consisting of: autoimmune encephalitis, paraneoplastic neurological syndromes, and inflammatory peripheral neuropathies.

Autoimmune encephalitis (AE) is an inflammatory syndrome where the immune system attacks healthy cells and tissues of the brain and/or spinal cord. Specific antibodies linked to this condition have been identified, such as anti-LGI1, Caspr2, NMDAR, GAD, AMPAR and GABA_{A/B}R antibodies. Some types of autoimmune encephalitis are caused by infection in which case the term ‘post-infectious encephalitis’ is used.

A paraneoplastic neurological syndrome (PNS) is a disorder caused by the presence of a tumor in the body, inducing an immune response against said tumoral cells. These autoantibodies are directed against tumoral antigens but also cross-react with neurons, and therefore might destroy cells of the nervous system. PNS are most commonly associated with cancers of the lung, breast, ovaries, or lymphoma. Several autoantibodies have been identified as being markers of such PNS; for example, the patent application WO 2019/211392 reports the identification of autoantibodies against TRIM9 and/or TRIM67 as biomarkers of PNS.

Peripheral neuropathies develop when nerves in the body's extremities, such as the hands, feet and arms, are damaged. Symptoms depend on the nature of the affected nerves. Inflammatory peripheral neuropathies may be of autoimmune origin, and in such cases may have associated autoantibodies.

More particularly, when the process for identifying patients affected by an autoimmune neurological disease is implemented, the effectively diagnosed autoimmune neurological disease is chosen among the group consisting of:
sensory neuronopathy,
limbic encephalitis,
cerebellar syndrome, and
other inflammatory peripheral neuropathies such as small fiber neuropathy and chronic inflammatory demyelinating polyneuropathy,
rhombencephalitis, and
opsoclonus-myoclonus.

Sensory neuronopathy (SNN) is characterized by a primary damage of the body of the neurons located at the dorsal root ganglia. The main clinical complains at onset are pain and paresthesia with usually asymmetric distribution that involves arms and legs. In some patients, pain may be prominent while in others numbness, limb ataxia, and pseudo-athetotic movements of the hands predominate.

Sensory neuronopathy is distinct of sensory-motor neuropathy affecting mostly a large proportion of persons aged of 70 years or more.

In a specific embodiment of the invention, the identified patients with the process of the invention are affected by autoimmune sensory neuronopathy.

Small fiber neuropathy (SFN) is characterized by a specific involvement of unmyelinated nerve fibers or their corresponding neurons. Pain is predominant and usually disabling and severe. Loss of pain and thermal sensation contrasts with the preservation of other sensations. The distribution may be length or non-length dependent.

Chronic inflammatory demyelinating polyneuropathy (CIDP) is characterized by demyelination and remyelination of peripheral nerves resulting in a mixture of motor and sensory disturbances with diffuse areflexia in the four limbs. Electroneuromyography is the main tool to demonstrate the demyelinating pattern of the neuropathy.

Limbic encephalitis (LE) describes the condition when limbic areas of the brain are inflamed and consequently not functioning properly. LE is characterized by subacute onset of confusion with marked impairment of short-term memory. Seizures are common and may antedate by months the onset of cognitive deficit.

Cerebellar syndrome is a form of ataxia originating in the cerebellum. Clinicians often use visual observation of people performing motor tasks in order to look for signs of ataxia in limbs, trunk, and gait. Ataxia may be accompanied of other features of cerebellar origin, such as dysarthria (slurred speech) and oculomotor disturbances.

Rhombencephalitis refers to inflammatory diseases affecting the hindbrain (brainstem and cerebellum) and has a wide variety of etiologies including infections, autoimmune diseases, and PNS. Rhombencephalitis is characterized by a wide range of clinical manifestations, such as altered level of consciousness, cranial nerve involvement, movement disorders, and ataxia.

Opsoclonus-myoclonus syndrome (OMS) is defined by the presence of spontaneous, arrhythmic and large amplitude conjugate saccades occurring in all directions of gaze, without saccadic interval. Opsoclonus is usually associated with myoclonus of the limbs and trunk, and occasionally with encephalopathy.

The present invention also concerns a process of classification of patients, comprising:
a. identifying among a group of patients affected with neurological signs and symptoms a subgroup of patients affected with an autoimmune neurological disease, wherein the step of identification is made by the process as defined previously, and
b. classifying said patients.

In particular, said patients will be classified among at least two subgroups:
(i) patients affected with neurological signs and symptoms having an autoimmune origin, and
(ii) patients affected with neurological signs and symptoms without any autoimmune origin.

The population of patients that is concerned by this classification process is composed of patients affected with neurological symptoms or diseases. The aim of the process is to identify, among these patients, those presenting a neurological disease of autoimmune origin. Advantageously, this subgroup of patients may then benefit from a specific therapeutic strategy, based on the administration of immunomodulatory compounds.

In another aspect, the present invention concerns a process of follow-up of a patient affected with an autoimmune neurological disease, comprising at least the following two successive steps:
a. Quantifying at least one type of AGO-Abs in a biological sample of said patient obtained at a time T1, according to a process as defined above;
b. Quantifying at least one type of AGO-Abs in a biological sample of said patient obtained at a time T2 posterior to T1, according to a process as defined above;
   wherein an increase of AGO-Abs quantity between steps a and b means a worsening of the clinical condition of the patient.

By contrast, a decrease of AGO-Abs quantity may indicate that the patient is likely to recover and/or a successful treatment.

As previously specified, the quantification of at least one type of AGO-Abs may be performed by any technique known by the person skilled in the art.

Advantageously, values of AGO-Abs quantity are compared with reference values, representative of a certain stage of the disease. In neurological diseases, the stages of disease are ranked accorded to the modified Rankin scale (mRS), that comprises stages 0 to 6, as presented in the examples section and in particular in table 2.

In another aspect, the present invention concerns a specific biomarker of the autoimmune nature of neurological diseases, consisting of AGO-Abs, in particular directed against at least one of the following proteins: AGO1, AGO2, AGO3, AGO4, and combinations thereof, as well as fragments thereof.

In a specific embodiment, AGO-Abs are mainly directed against AGO1 and AGO2, but AGO-Abs usually recognize AGO3 and AGO4 as well.

This biomarker is highly specific of the autoimmune nature of neurological diseases: as shown in example 3, only two serum samples and no CSF sample, over 754 tested control samples, were positive for AGO-Abs.

In another aspect, the present invention concerns a kit for the implementation of a process as defined above, comprising means for the detection and/or quantification of at least one type of AGO-Abs in a biological sample of an individual.

In particular, said kit comprises:
a. At least one antigen or antigen-expressing cell or nucleic acid coding for an antigen, chosen among the group consisting of:
   i. Cells overexpressing selectively AGO1, AGO2, AGO3, or AGO4 proteins, or a variant thereof, or any combination thereof;
   ii. AGO1, AGO2, AGO3 or AGO4 protein, or a variant thereof, or any combination thereof;
   iii. A fragment or peptide issued from AGO1, AGO2, AGO3, or AGO4 protein, or a variant thereof, or any combination thereof;
   iv. An expression vector carrying at least one nucleic acid encoding AGO1, AGO2, AGO3, or AGO4 protein, or a variant thereof, or any fragment or peptide issued from AGO1, AGO2, AGO3, or AGO4 protein, or a variant thereof, or any combination thereof;
b. Anti-human immunoglobulin antibody coupled to a probe; and
c. Reactant(s) useful for the in vitro step of detection and/or quantification.

Useful probes, also referred herein as labels, are well known by the person skilled in the art; for example, probes may be chosen among fluorescence markers, e.g. Alexa488, Alexa555; or enzymes, e.g horse-radish peroxidase.

Other probe or labels may be selected from the group comprising radioactive, chemiluminescent or enzymatically active labels. Exemplary labels are described in (Obermaier et al., 2021).

Preferably the expression vector comprises a promotor controlling the expression of the nucleic acid carried, preferably an inducible promotor.

The person skilled in the art is familiar with the preparation of cells, reagents and methods, in particular with a view to detection of an autoantibody using immunofluorescence.

In another aspect, the present invention concerns a solid carrier coated with one or more compound from the group comprising: a polypeptide comprising AGO1 or a variant thereof, a polypeptide comprising AGO2 or a variant thereof, a polypeptide comprising AGO3 or a variant thereof and a polypeptide comprising AGO4 or a variant thereof.

In addition, the carrier may comprise one or more compound from the group comprising the following antigens: FGFR3, Hu, Yo, Ri, CV2, PNMA1, PNMA2, DNER/Tr, ARHGAP26, ITPR1, ATP1A3, NBC1, Neurochrondrin, CARPVIII, Zic4, SOX1, Ma, MAG, MPO, MBP, GAD65, amphiphysin, recoverin, GABA A receptor, GABA B receptor, glycine receptor, gephyrin, IgLON5, DPPX, aquaporin-4, MOG, NMDA receptor, AMPA receptors, GRM1, GRM5, LGI1, VGCC, mGluR1, CASPR2, ATP1A3, also referred to as alpha 3 subunit of human neuronal Na(+)/K(+) ATPase and Flotillin1/2.

In a preferred embodiment, the carrier may be selected from the group comprising: a glass slide, a biochip, a microtiter plate, a lateral flow device, a test strip, a membrane (preferably for line blot or western blot), a chromatography column and a bead, such as a magnetic or fluorescent bead, and is preferably chosen among the group consisting of:

a microtiter plate and a glass slide for immunofluorescence. This carrier may be part of a kit according to the present invention.

In another aspect, the present invention concerns a process of treatment of an autoimmune neurological disease in patients in need thereof, comprising the following steps:
a) classifying patients affected with neurological diseases according to the process of classification described above, and
b) administering an appropriate immunomodulatory compound to a subgroup of patients identified as being affected with an autoimmune neurological disease.

Traditional therapies for autoimmune disease rely on administration of immunomodulatory compounds, generally immunosuppressive, for alleviating the manifestations of the diseases. In particular, immunotherapeutic compounds such as corticosteroids, intravenous immunoglobulin, plasmapheresis, cyclophosphamide, rituximab, mycophenolate mofetyl or all other molecules able to modulate lymphocytes function are used.

Naturally, an effective amount of this immunomodulatory compound will be administered to the patients belonging to the defined subgroup.

As is shown in the examples section, nearly 85% of the patients positive for AGO-Abs, and treated with immunomodulatory compounds, presented an improved or stable condition at the last follow-up.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Materials and Methods

Two distinct approaches were used to identify the autoantibodies and their antigen.

In a first approach, the CSF of a patient with limbic encephalitis (patient XI), which showed an atypical staining on immunohistochemistry, was used to perform immunoprecipitation and mass spectrometry. In parallel, protein micro-arrays were used for autoantibody characterization in serum of patients with peripheral neuropathies. Finally, the specificity of the identified target was confirmed by CBA, and immunoadsorption; epitope mapping was also performed.

Patients Samples

Patient's serum and CSF were obtained from the biobank NeuroBioTec (Hospices Civils de Lyon BRC, France, AC-2013-1867, NFS96-900). Written consent was obtained from all patients. The Institutional Review Board of the University Claude Bernard Lyon 1 and Hospices Civils de Lyon, and the CHU of Saint-Etienne approved the study, which has been carried out in accordance with the Code of Ethics of the World Medical Association (Declaration of Helsinki).

For the CBA study, 250 CSF samples were selected from patients with suspected AE/PNS and 42 sera of patients with peripheral neuropathies. As controls, 312 CSF and 442 sera were selected from various patients with or without neurological symptoms (see table 1). All samples were collected from October 2007 to December 2019.

For the first ELISA study (example 6), the sera of 688 subjects were used: 277 with neuropathy (126 SNN), 67 small fiber neuropathies (SFN), 84 other peripheral neuropathies (OPN)), 173 with central nervous system diseases (CNSD), 122 with autoimmune diseases (AID), and 116 healthy controls (HC).

A second ELISA study has been realized (examples 7 and 8) with the sera of 823 subjects: 433 with neuropathy, comprising 132 SNN, 80 small fiber neuropathies (SFN), 116 chronic inflammatory demyelinating polyneuropathy, and 105 other peripheral neuropathies (OPN), as well as 274 with systemic autoimmune diseases (AID), comprising 87 with SLE, 146 with SjS, and 41 other autoimmune diseases, as well as 116 healthy controls (HC).

Immunohistochemistry

Freshly prepared adult rat brains were fixed in 4% paraformaldehyde (PFA) for 1 h, frozen, and sliced into 12 μm-thick sections. Immunolabeling was performed using patient's CSF (1:10) and revealed with Alexa 488 fluorophore-conjugated secondary antibodies (1:1 200, A11013, Thermo Fischer, Courtaboeuf, France).

Immunoprecipitation and Mass Spectrometry-Based Identification

The CSF of a patient with limbic encephalitis and atypical staining by immunohistochemistry (patient XI) was used, as well as a control CSF (without staining pattern), to identify the target by immunoprecipitation and mass spectrometry. Five μL of CSF were mixed with 50 μL of protein G-conjugated agarose beads (Sigma Aldrich, Lyon, France) and completed to 500 μL with PBS. The mixture was incubated for 2 h at 4° C. with rotation to allow for the coupling of CSF antibodies with protein G. Simultaneously, whole protein extract from one rat brain was prepared and incubated with 50 μL of agarose beads. Nonspecific contaminant was removed from the lysate by 5 minutes centrifugation at 16,000 g and 4° C. Cleared lysate was subsequently used for immunoprecipitation with antibodies-conjugated agarose beads. Immunoprecipitate was analyzed by SDS-PAGE, followed by silver-staining, Western blotting, and mass spectrometry-based proteomics, as previously described (Casabona et al., 2013) Briefly, proteins were in-gel digested using modified trypsin (Promega, sequencing grade), and resulting peptides were analyzed by online nanoLC-MS/MS (UltiMate 3000 and LTQ-Orbitrap Velos Pro, Thermo Scientific). Peptides and proteins from different samples were identified, filtered, and compared using Mascot (version 2.6.0, Matrix Science) and Proline software (version 2.0).

Protein Microarrays

Sera of 34 patients with peripheral neuropathies (12 with sensory neuronopathy, 22 with other peripheral neuropathies) and 9 healthy controls were used to perform protein microarray. Sera were prepared and then tested on HuProt 31™ microarrays (CDI labs, Baltimore, MD, USA). The arrays were blocked for 16 h at 4° C. in blocking buffer (2% bovine serum albumin in 0.05% Tween-20), sera were incubated for 2 h at room temperature as a 1:1,000 dilution in blocking buffer. Unbound immunoglobulin (Ig) were washed away with 0.05% Tween-20 in phosphate buffer saline. Bound IgG were revealed with Alexa546-labelled polyclonal goat anti-human IgG(H+L) antibodies (Invitrogen, Cat. A21089, Renfrew, UK) incubated for 2 h at room temperature. The slides were scanned with a Tecan L5400 microarray scanner and the images analyzed with GenePix Pro. Subsequent data analysis included local background subtraction, deriving average and CV % of duplicate spots, and subtraction of unspecific signal resulting from secondary antibody (assessed by probing a serum-free array). Proteins with CV %>25% in their duplicate spots (technical replicates) were excluded from the analysis. The data set was standardized for each subject to normalize for systematic biases between the samples (e.g., systematic labelling efficiency differences). Standard normalization between subjects (z-score statistics) was used to compare the reactivities of each antigen among the samples. A z-score cut-off of 4 standard deviations above the controls and a specificity of 100% (i.e., absent in all controls) were applied to identify antigen candidates.

Cell-Based Assay

A CBA was performed to screen a large cohort of samples for AGO-Abs. HEK 293 cells were transfected with VP5-HA-AGO1 (SEQ ID NO. 1), VP5-HA-AGO2 (SEQ ID NO. 2), VP5-HA-AGO3 (SEQ ID NO. 3), VP5-HA-AGO4 (SEQ ID NO. 4), VP5-HA-TNRC6A, VP5-HA-TNRC6B, or VP5-HA-TNRC6C plasmid for a transient overexpression. VP5 is the plasmid, HA is a molecular tag, and TNRC6 (trinucleotide repeat containing protein) A, B, and C are proteins involved in RNA degradation and present in GW bodies, like AGO proteins.

Fixed and permeabilized cells were immunostained with patients CSF (1:10) or serum (1:100), and then revealed with Alexa 555 fluorochrome-conjugated secondary antibodies (1:1 200, A21433, Invitrogen). Photographs of stained cells were taken with fluorescent microscope Axio Imager Z1 (Carl Zeiss, Marly le Roi, France). For double immunolabelling, rabbit anti-AGO2 (1:1 000) or rabbit anti-HA (1:1 000, P1985, Fisher Scientific) was added to dilute CSF and was revealed by Alexa488-conjugated secondary antibody (1:1 000, A11034, Invitrogen). Antibody titres were obtained by using serial dilutions of serum (if available) and CSF on HEK 293 cells expressing either AGO1 or AGO2. Patients IgG subtypes contained in serum and CSF (if available) were identified using AGO1 or AGO2-transfected HEK 293 cells and secondary anti-human antibodies specific for IgG1 (1:1 000, MCA4774, Bio-Rad, Marnes-la-Coquette, France), IgG2 (1:500, 555873, BD Biosciences, Le Pont de Claix, France), IgG3 (1:1 000, 5247-9850, Bio-Rad), or IgG4 (1:500, 555881, BD Biosciences). Bound IgG were revealed by a goat anti-mouse IgG antibody coupled with Alexa555 (1:1 000, A21424, Invitrogen).

Enzyme-Linked Immunosorbent Assay (ELISA)

Sera were screened via ELISA in order to detect AGO1-Abs and AGO2-Abs antibodies and IgG isotypes. Detection of AGO1-Abs and AGO2-Abs was performed by using a previously described indirect ELISA (Moritz et al., 2019). The human recombinant proteins AGO1 (11225-H07B, Sino Biological, Eschborn, Germany) and AGO2 (11079-H07B, Sino Biological) were used for antigen coating. Wells were either coated with 1 μg/mL protein or mock-coated, washed twice with 300 μL washing solution (0.1% Tween-20 in PBS), and blocked with blocking solution (0.06% Tween-20, 0.1% fish gelatin, and 3% bovine serum albumin (BSA) in phosphate-buffered saline (PBS). Sera were diluted 1:100 in blocking solution and incubated 16 h at 4° C. After 6× washing with washing solution, 1:3,000 (i.e., 0.43 μg/L) of rabbit-anti-human IgG (Dako, Glostrup, Denmark) in the blocking buffer was incubated for 2 h at 4° C. After 10× washing with washing buffer followed 30 min incubation with 0.4 mg/mL of 0-phenylenediamine dihydrochloride in phosphate/citrate buffer (0.05 mM, pH 5, Sigma-Aldrich, Saint-Quentin Fallavier, France). Optical density (OD) was measured at 450 nm with Multiscan EX Elisa Reader (BMG Labtech, Champigny sur Marne, France). For each serum, the signals were normalized for the serum-specific background noise by calculating the difference between the optical density of the AGO-coated well and that of the non-coated well (ΔOD) (Moritz et al., 2019). The test was considered positive when the difference was 4 standard deviations (SD) above the average signal of 116 healthy blood donors (z-score>4). To determine IgG isotypes, indirect ELISA was repeated with secondary anti-human antibodies specific against IgG1, IgG2, IgG3, or IgG4 (Tholance et al., 2020).

AGO1 and AGO2-Abs Immunoadsorption

A total of 1.6×10⁶ HEK 293 cells were seeded in 6-wells plate. Cells were transfected with AGO1 or AGO2 by using LTX (10573013, Fisher Scientific) following manufactory's instruction. After 24 h, cells were fixed with 4% PFA. Then, 1 mL of patient XI's CSF (1:100) was incubated in each well for 24 h until no more CBA signal was observed. Diluted CSF was then used for immunohistochemistry on rat brain section as described above. Control experiment was performed in the same way on non-transfected HEK 293 cells.

### Example 1. Identification of Argonaute (AGO) Proteins as Targets of Autoantibodies in Neurological Diseases

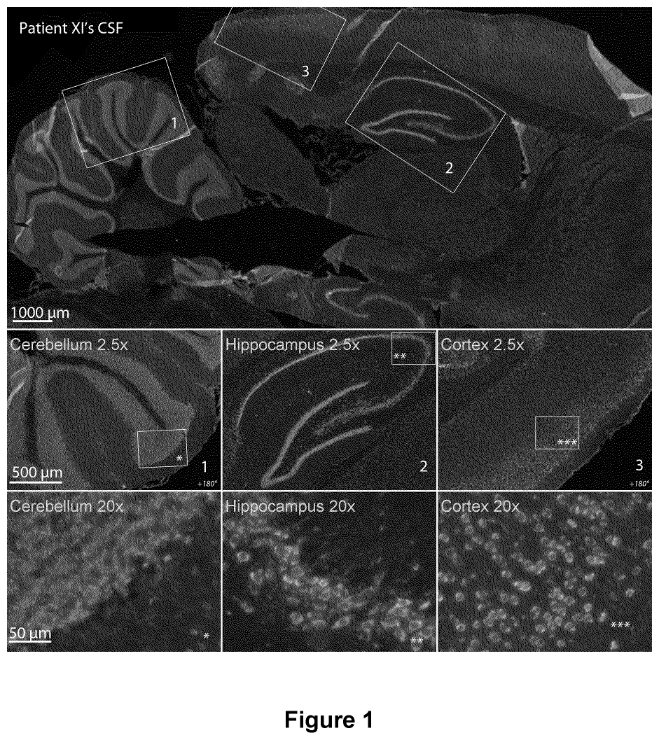

AGO proteins were identified as targets of autoantibodies using two different approaches. First, among a series of CSF of patients with suspected AE/PNS, one patient CSF (patient XI) showed reactivity with the cytoplasm of neurons of the hippocampus (granular neurons of the dentate gyrus and CA1, CA3 pyramidal cells), cerebellum (granular cells, some cells in the molecular layer), and cerebral cortex, which was different from known autoantibodies associated with AE (FIG. 1).

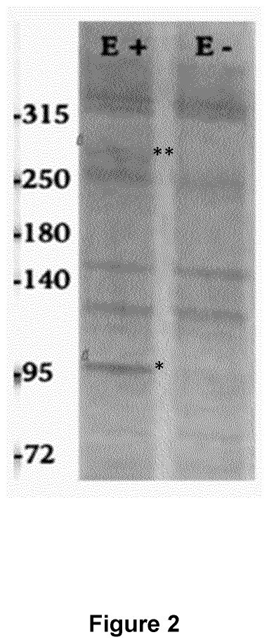

Using whole rat brain homogenates and immunoprecipitation (FIG. 2), a 100 kDa band was observed with patient XI's CSF but not with control CSF (FIG. 2 *). Subsequent mass spectrometry analyses revealed AGO1 and AGO2 as the most highly enriched proteins in the immunoprecipitate. Other AGO proteins such as AGO3 and AGO4 were also specifically enriched with patient XI's CSF as well as TRNC6 protein.

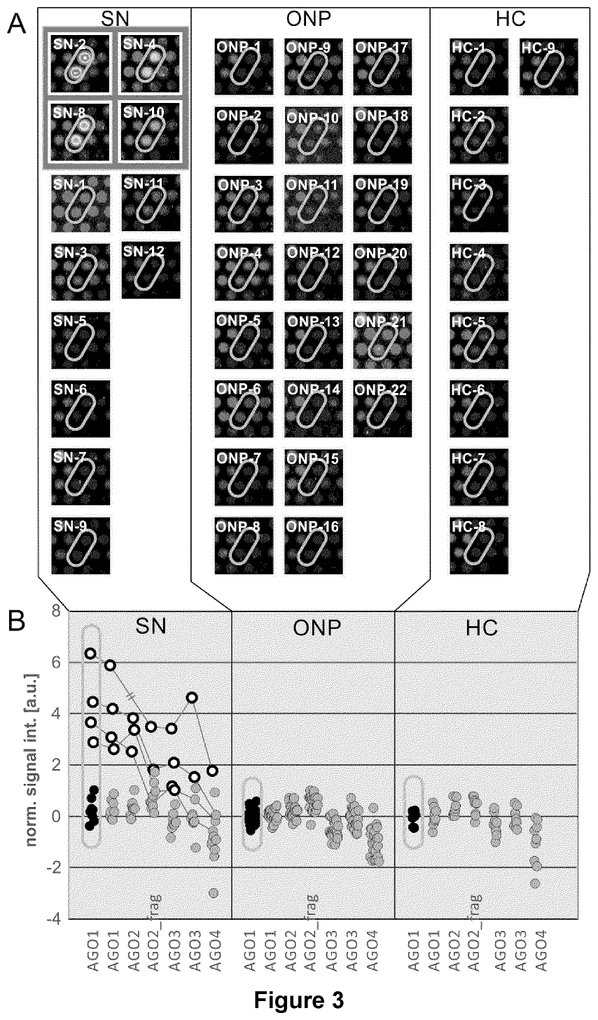

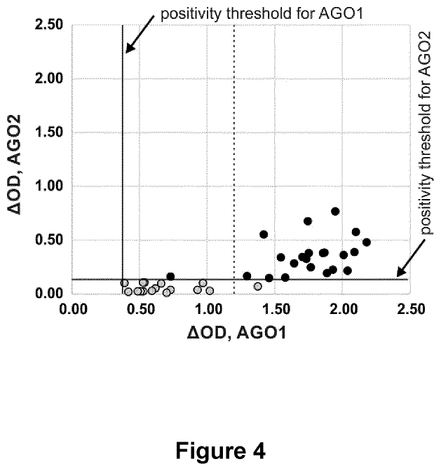

Using a different approach, sera of patients with peripheral neuropathies were screened by protein microarrays, and revealed that AGO1 and AGO2 proteins were significantly targeted by 4 and 3 patients, respectively, out of 12 sensory neuronopathy patients with a suspected autoimmune origin, but none of the controls (FIGS. 3 and 4). AGO3 and AGO4 proteins were significantly targeted by 1 and 3 of the same patients, but with lower reactivities (FIG. 3).

### Example 2. Identification of Neurological Patients with AGO-Abs

To identify new patients and to validate the specificity of AGO-Abs, different CBA were constructed, each using one of the AGO plasmids as described above.

HEK 293 cells were transfected with VP5-AGO1-4 or VP5-TNRC6B plasmid, for a transient overexpression. Fixed and permeabilized cells were then immunostained with anti-HA antibody and patient XI's CSF.

Patient XI's CSF reacted with AGO1 to 4-transfected cells while a control CSF did not and no signal was observed on non-transfected cells (data not shown). No signal was observed in HEK-293 cells transfected with TRNC6. Furthermore, after anti-AGO1 and anti-AGO2 antibodies depletion, patient XI's CSF did not react anymore on immunohistochemistry, confirming that patient XI's CSF contained AGO-Abs and that the staining pattern in FIG. 1 was caused solely by antibodies targeting AGO proteins. Serum of patient XI contained also AGO-Abs and likewise, three out of four samples of patients with sensory neuronopathy positive by protein microarray specifically bind AGO1 and AGO2-HEK293-transfected cells (patient I, II and III; data not shown).

In order to identify other patients with AGO-Abs, 250 CSF of patients with suspicion of AE/PNS and the sera of 42 patients with peripheral neuropathies were retrospectively screened by CBA. Among them, 12 additional patients with AGO-Abs were identified.

In addition, 3254 CSF sent to the French reference center for suspicion of AE or PNS were prospectively screened for AGO-Abs immunohistochemical pattern during six months (between Aug. 28, 2019 and Feb. 25, 2020). 5 new cases with AGO-Abs were identified. During the same period, the following autoantibodies were identified in these 3254 CSF: 21 NMDAR, 32 Lgi1, 15 Hu, 8 CASPR2, 8 Yo, 7 CV2/CRMP5, 2 AK5 and 2 AMPAR autoantibodies.

### Example 3. Specificity of AGO-Abs

To establish the specificity of AGO-Abs, a total of 754 control samples of patients with or without neurological diseases and without suspicion of AE or PNS were screened: 312 CSF and 442 sera (table 1). Among these controls, only two serum samples and no CSF were positive for AGO-Abs. One of the two patients with a AGO-Abs-positive serum had a small cell lung cancer without neurological symptoms and the other one had several autoimmune systemic diseases (Sjögren syndrome, autoimmune thyroid disease, rheumatoid arthritis, and idiopathic thrombocytopenia) and multiple autoantibody specificities (anti-Sjögren syndrome-related antigen A [SSA], anti-Sjögren syndrome-related antigen B [SSB], anti-thyroid peroxidase, anti-nuclear, anti-liver mitochondrial, anti-cyclic citrullinated peptide 2) without neurological symptoms.

In conclusion, this biomarker is highly specific of the autoimmune nature of neurological diseases, as only 2 over 754 tested control samples were positive.

TABLE 1
Control samples tested via CBA for AGO-Abs

| | | Positive |
|---|---|---|
| Control subgroup | Sample type | samples n/N |
| Undiagnosed neurological | CSF | 0/91 |
| syndromes | | |
| Undiagnosed neurological | Serum | 0/195 |
| syndromes | | |
| Alzheimer's disease | CSF | 0/90 |
| Fronto-temporal dementia | CSF | 0/25 |
| Lewy body dementia | CSF | 0/15 |
| Psychosis | CSF | 0/20 |
| Normal pressure | CSF | 0/15 |
| hydrocephalus | | |
| Patients without neurological | CSF | 0/56 |
| symptoms | | |
| Healthy controls | Serum | 0/17 |
| Lung cancer, neurological | Serum | 1/166 |
| asymptomatic | | |
| Autoimmune diseases | Serum | 1/64 |
| without neurological | | |
| involvement | | |
| TOTAL OF CONTROLS | CSF | 0/312 |
| | Sera | 2/442 |

### Example 4. Clinical and Paraclinical Characteristics of AGO-Abs Positive Neurological Patients

A total of 21 patients with neurological symptoms with AGO-Abs detected in serum and/or CSF samples was identified: 15/21 (71.4%) are women, with a median age of 57 years (range 25-85).

Patient features are presented in table 2.

TABLE 2
Clinical characteristics of 21 patients with AGO-antibodies

| | | | | | | | | | mRS at onset |
|---|---|---|---|---|---|---|---|---|---|
| | | | | | | | | | →mRS at |
| | | | | | | Auto- | | | last |
| | Sex, | | | | | immune | | | follow-up |
| Patient | age | Neurological | Ancillary | | Other | co- | | | (delay in |
| no | (y) | presentation | tests | CSF | Ab | morbidities | Cancer | Treatment | months) |
| I | M, | Sensory | EMG + SNN | NA | No | Sarcoidosis | No | IVIG + steroids + | NA |
| | 54 | neuronopathy | | | | | | PEX + CP + | |
| | | | | | | | | RTX + AZA + MTX | |
| II | F, | Sensory | EMG + SNN | NA | ANA, SSA | Sjögren | No | Steroids + IVIG + | 3 → 3 (144) |
| | 40 | neuronopathy | | | | syndrome | | CP | |
| III | F, | Sensory | EMG + SNN | NA | SSA, SSB | Sjögren | No | NA | NA |
| | 53 | neuronopathy | | | | syndrome | | | |
| IV | M, | Sensory | EMG + SNN | 0.7 g/L | No | No | No | No | 2 → 3 (5) |
| | 74 | neuronopathy | | proteins, no | | | | | |
| | | | | cells, OCB − | | | | | |
| V | F, | Sensory | EMG + SNN | 0.42 g/L | No | No | Colon | IVIG | 2 → 2 (2) |
| | 57 | neuronopathy | | proteins, no | | | Adenocar- | | |
| | | | | cells, OCB − | | | cinoma | | |
| VI | F, | Bilateral | EMG + SNN; | 1.22 g/L | ANA, SSA | Sjögren | Breast | PEX + IVIG + | 2 → 1 (22) |
| | 56 | asynchronous | MRI contrast | proteins, 12 | | syndrome, | cancer | steroids | |
| | | trigeminal | enhancement | cells/mm³ | | vitiligo | | | |
| | | neuralgia; 2 | of both | | | | | | |
| | | years later, | trigeminal | | | | | | |
| | | sensory | nerves | | | | | | |
| | | neuronopathy | | | | | | | |
| VII | F, | Sensory | EMG + SNN | NA | SSA, SSB | No | No | CP + RTX | 3 → 3 (3) |
| | 55 | neuronopathy | | | | | | | |
| VIII | F, | Sensory | EMG + SNN | 0.43 g/L, 1 | ANA | Sjögren | No | Steroids + AZA | 4 → 3 (156) |
| | 54 | neuronopathy | | cell/mm³ | | syndrome | | | |
| IX | M, | Sensory | EMG | NA | ANA, MAG | No- | No | RTX | 2 → 1 (168) |
| | 79 | demyelinating | demyelinating | | | | | | |
| | | polyneuropathy | abnormalities | | | | | | |
| | | | in four limbs | | | | | | |
| X | M, | Sensory-motor | EMG axonal | 0.35 g/L, 1 | No | No | No | No | 2 → NA |
| | 71 | axonal | abnormalities | cell/mm³ | | | | | |
| | | polyneuropathy | in four limbs | | | | | | |
| XI | F, | Limbic | MRI bilateral | 0.23 g/L | No | Sjögren | No | IVIG | 2 → 0 (29) |
| | 78 | encephalitis: | hippocampal | proteins, 5 | | syndrome | | | |
| | | amnesia, | atrophy; EEG − | cells/mm³, | | | | | |
| | | psychiatric | | OCB + | | | | | |
| | | symptoms | | | | | | | |
| | | Movement | | | | | | | |
| | | disorders | | | | | | | |
| XII | F, | Limbic | MRI | 0.5 g/L | SSA | No (Minor | No | Steroids + IVIG | 5 → 2 (9) |
| | 25 | encephalitis: | bitemporal | proteins, 84 | | salivary | | | |
| | | seizures, | FLAIR | cells/mm³, | | gland biopsy | | | |
| | | amnesia, | abnormalities; | OCB + | | negative) | | | |
| | | psychiatric | EEG + | | | | | | |
| | | symptoms | | | | | | | |
| XIII | F, | Limbic | MRI | 0.38 g/L | No | Cutaneous | No | Steroids + IVIG | 3 → 2 (2) |
| | 41 | encephalitis: | bitemporal | proteins, 2 | | lupus with | | | |
| | | seizures, | FLAIR | cells/mm³, | | arthritis | | | |
| | | amnesia, | abnormalities; | OCB − | | | | | |
| | | psychiatric | EEG + | | | | | | |
| | | symptoms | | | | | | | |
| XIV | F, | Limbic | MRI right | 0.91 g/L | GABA_{B}R | No | SCLC | Steroids + IVIG | 5 → 6 (8) |
| | 74 | encephalitis: | temporal | proteins, 15 | | | | | |
| | | seizures | FLAIR | cells/mm³, | | | | | |
| | | (status), | abnormality; | OCB + | | | | | |
| | | amnesia, | EEG + | | | | | | |
| | | psychiatric | | | | | | | |
| | | symptoms | | | | | | | |
| XV | F, | Limbic | MRI left | Normal | NA | No | No | Steroids | 3 → 1 (0.5) |
| | 85 | encephalitis: | temporal | proteins, 8 | | | | | |
| | | seizures, | FLAIR | cells/mm³ | | | | | |
| | | amnesia | abnormality; | | | | | | |
| | | | EEG + | | | | | | |
| XVI | F, | Limbic | MRI right | 0.47 g/L | NA | No | No | No | 5 → 6 (1.5) |
| | 67 | encephalitis: | temporal | proteins, 2 | | | | | |
| | | seizures | FLAIR | cells/mm³ | | | | | |
| | | (status), | abnormality; | | | | | | |
| | | amnesia, | EEG + | | | | | | |
| | | psychiatric | | | | | | | |
| | | symptoms | | | | | | | |
| XVII | F, | Subacute | MRI left | 0.7 g/L | SSA | No (Minor | No | Steroids + IVIG | 2 → 4 (37) |
| | 30 | cerebellar | hemicerebellar | proteins, 465 | | salivary | | | |
| | | syndrome | hypersignal | cells/mm³, | | gland biopsy | | | |
| | | | → cerebellar | OCB + | | negative) | | | |
| | | | atrophy | | | | | | |
| XVIII | M, | Chronic | MRI cerebellar | Normal, | No | No | Epidermoid | No | 4 → NA |
| | 67 | cerebellar | atrophy | OCB − | | | carcinoma | | |
| | | syndrome | | | | | | | |
| XIX | F, | Rhomben- | MRI brainstem | 0.9 g/L | ANA, SSA, | Autoimmune | No | Steroids | 2 → 0 (2) |
| | 35 | cephalitis: | FLAIR | proteins, 424 | GAD, | hepatitis, | | | |
| | | headache, | abnormality [symbol] | cells/mm³ | anticardio- | Sjögren | | | |
| | | vertigo, | LETM imaging; | | lipine | syndrome | | | |
| | | vomiting, | EEG − | | | | | | |
| | | nistagmus, | | | | | | | |
| | | gaze-palsy | | | | | | | |
| XX | M, | Opsoclonus- | CT normal; | Normal | No | No | Lingual | No | 3 → NA |
| | 58 | myoclonus | EEG − | proteins, no | | | carcinoma | | |
| | | | | cells, OCB − | | | | | |
| XXI | F, | Transitory | MRI normal; | 1 g/L | SSA, SSB | No | No | No | 5 → 4* (1) |
| | 76 | low level of | EEG − | protein, 14 | | | | | |
| | | consciousness | | cells | | | | | |

*Previous paraplegia due to compressive myelopathy
EEG−, normal; EEG+, temporal epileptic abnormalities.
EMG + SNN, at least 1 sensory action potential (SAP) absent or SAP <30% of the lower limit of normal in the upper limbs, and no more than 2 nerves with abnormal motor nerve conduction studies in the lower limbs, according to previously published criteria.
Abbreviations: Ab, autoantibodies; ANA, anti-nuclear antibody; ATD, autoimmune thyroid disease; CSF, cerebrospinal fluid; CT, computerized tomography; EEG, electroencephalography; EMG, electromyography; F, female; FLAIR, fluid-attenuated inversion recovery; GABA_{B}R, gamma-amino butyric acid receptor B; GAD, glutamic-acid decarboxylase; ITP, idiopathic thrombocytopenic purpura; IVIG, intravenous immunoglobulin; LETM, longitudinally extensive transverse myelitis; M, male; MAG, myelin-associated glycoprotein; MRI, magnetic resonance imaging; mRS, modified Rankin scale; NA, not available; OCB, oligoclonal bands; PEX, plasma exchange; RA, rheumatoid arthritis; SCLC, small-cell lung cancer; SNN, sensory neuronopathy; SSA, Sjögren syndrome related antigen A; SSB, Sjögren syndrome related antigen B; y, years-old.

Among these patients, the most common clinical presentation was sensory neuronopathy (SNN; 8/21, 38.1%), followed by limbic encephalitis (LE; 6/21, 28.6%), cerebellar syndrome (2/21, 9.5%), other peripheral neuropathies (2/21, 9.5%), rhombencephalitis (1/21, 4.8%), and opsoclonus-myoclonus (1/21, 4.8%).

One patient (1/21, 4.8%) presented with a transitory low level of consciousness that was not described in detail in the charts.

When analyzed, the CSF was inflammatory (pleocytosis 5 cells) in 8/16 (50.0%) patients; oligoclonal bands were positive in 4/9 (44.4%) patients.

All patients with central nervous system involvement, excepting the patient with transient impairment of consciousness, had brain MRI abnormalities, such as temporal or cerebellar hyperintensities or atrophy. The patient presenting with opsoclonus-myoclonus had a normal cranial computerized tomography. (see table 2)

Cancer was diagnosed in 5/21 (23.8%) patients: a small-cell lung cancer (SCLC) in a patient also positive for antibodies against the gamma-amino butyric acid receptor B (GABA_{B}R-Abs); a breast cancer in remission diagnosed 2 years before the neurological symptoms in a patient with cranial nerve involvement and SNN (9 lumbar punctures did not find malignant cells); a colon adenocarcinoma detected during the tumor screening performed after SSN diagnosis; a epidermoid carcinoma in remission for 3 years in a patient with a chronic cerebellar syndrome; and a lingual carcinoma diagnosed 5 years before but with a local recurrence only 4 months before the development of the neurological symptoms in the patient presenting with opsoclonus-myoclonus.

Autoimmune comorbidities (Sjögren syndrome in 6/21 patients, 28.6%) and/or co-occurring autoantibodies (mainly anti-SSA, detected in 8/21 patients, 38.1%) were present in 14/21 (66.7%) patients; whereas in the remaining 7 patients (7/21, 33.3%) AGO-Abs were the sole biomarker of autoimmunity.

Fourteen (14/21, 66.7%) patients received immunotherapy, including steroids (n=10), intravenous immunoglobulin (n=9), cyclophosphamide (n=3), rituximab (n=3), plasma exchange (n=2), azathioprine (n=2), and methotrexate (n=2). Median mRS at onset was 3 (range 2-5) for the 19 patients with available information; median mRS at last follow-up for 16 patients was 2.5 (range 0-6); follow-up was not available for 5 patients.

Among the patients treated by immunotherapy with enough data regarding the evolution (n=13), 8/13 (61.5%) improved (lower mRS at last follow-up compared to that one at onset), 3/13 (23.1%) was stable (unchanged mRS), and 2/13 (15.4%) worsened.

Overall, two (2/21, 9.5%) patients died: patient XIV due to progression of her oncologic disease (SCLC with co-occurring GABA_{B}R-Abs), and patient XVI because of several complications of hepatic cirrhosis of alcohol-origin.

In conclusion, nearly 85% of the patients treated by immunotherapy improved or were stable at last follow-up, reinforcing the hypothesis of an immune mechanism.

### Example 5. Characteristics of AGO-Abs

All positive samples reacted with AGO1 and AGO2, and less strongly with AGO3 and AGO4. Serum titers of AGO-Abs ([200:2 560 000], median=40 000) were higher than those from the CSF ([10:512 000], median=400). Excepting or patient II and III, AGO1- and AGO2-Abs titers were higher than those of AGO3- and AGO4-Abs, in both serum and CSF (table 3 below).

Regarding the analysis of IgG subclasses in serum and CSF, all patients had essentially IgG1, but other IgG isotypes (i.e., IgG2, IgG3, and IgG4) were also found.

TABLE 3
AGO-Abs titers in CSF and serum

| Patient no | Sample | AGO1 | AGO2 | AGO3 | AGO4 |
|---|---|---|---|---|---|
| I | CSF | NA | NA | NA | NA |
| | Serum | 2 048 000 | 512 000 | 40 000 | 32 000 |
| II | CSF | NA | NA | NA | NA |
| | Serum | 256 000 | 256 000 | 1 536 000 | 40 000 |
| III | CSF | NA | NA | NA | NA |
| | Serum | 400 | 2 000 | 4 000 | − |
| IV | CSF | >100 | >100 | NA | NA |
| | Serum 1 | 32 000 | 40 000 | 16 000 | − |
| | Serum 2 | 96 000 | 128 000 | 24 000 | − |
| V | CSF | 1 600 | 800 | 500 | 250 |
| | Serum | 320 000 | 256 000 | 24 000 | − |
| VI | CSF | 3 200 | 1 600 | 1 600 | 100 |
| | Serum | 320 000 | 320 000 | 20 000 | <10 000 |
| VII | CSF | NA | NA | NA | NA |
| | Serum | 96 000 | 192 000 | 4 000 | 4 000 |
| VIII | CSF | NA | NA | NA | NA |
| | Serum | 192 000 | 192 000 | 4 000 | 4 000 |
| IX | CSF | NA | NA | NA | NA |
| | Serum | 512 000 | 192 000 | 8 000 | 200 |
| X | CSF | NA | NA | NA | NA |
| | Serum | 16 000 | 16 000 | 4 000 | 1 000 |
| XI | CSF | 12 800 | 51 200 | 400 | 400 |
| | Serum | 1 228 800 | 2 048 000 | 128 000 | − |
| XII | CSF | 200 | 200 | 60 | − |
| | Serum | 16 000 | 40 000 | 8 000 | − |
| XIII | CSF | NA | NA | NA | NA |
| | Serum | NA | >100 | NA | NA |
| XIV | CSF | NA | >100 | NA | NA |
| | Serum | NA | NA | NA | NA |
| XV | CSF | >100 | 200 | >100 | >100 |
| | Serum | NA | NA | NA | NA |
| XVI | CSF | >100 | 200 | >100 | − |
| | Serum | NA | NA | NA | NA |
| XVII | CSF | >100 | 1 600 | >100 | >100 |
| | Serum | NA | NA | NA | NA |
| XVIII | CSF | 10 | 60 | − | − |
| | Serum | NA | NA | NA | NA |
| XIX | CSF | >6 400 | >100 | >100 | >100 |
| | Serum | NA | NA | NA | NA |
| XX | CSF | 3 200 | 400 | 600 | − |
| | Serum | 96 000 | 24 000 | 16 000 | − |
| XXI | CSF | 1 200 | 800 | 30 | 120 |
| | Serum | NA | NA | NA | NA |

Abbreviations:
CSF, cerebrospinal fluid;
NA, not available;
+: positive without titer;
−: negative

### Example 6. Detection of AGO1-Abs and AGO2-Abs in Neurological Diseases Via ELISA

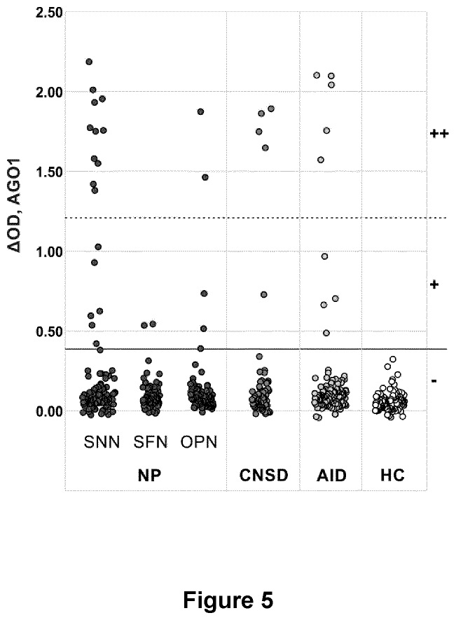

Using 13 AGO1-Abs-positive and 12 AGO2-Abs-positive sera detected via CBA, an ELISA test was implemented to detect AGO-Abs. ELISA tests detected 30 additional AGO1-Abs-positive samples and 10 additional AGO2-Abs-positive samples, suggesting a possible higher sensitivity for ELISA compared to CBA. ELISA using AGO2 antigen was much less sensitive, as their differences of optical density (ΔODs) were systematically lower than for AGO1 (FIG. 4 and FIG. 5).

### Conclusions

Although AGO-Abs reacted against all four AGO proteins, AGO1 and AGO2 were the main targets based on the results of the antibody titration, protein microarrays, and immunoadsorption. This shared reactivity has been linked to the high homology among the four AGO proteins (reaching 80%), though an epitope spreading phenomenon might also explain this finding.

AGO-Abs seems to be a specific biomarker of autoimmunity: only one sample out of the 754-screened controls was positive without evidence of neurological or systemic autoimmune disease.

### Example 7. Detection of Conformation-Specific AGO1 Abs Via an Adapted ELISA Approach

In parallel to the CBA assay, a standardized ELISA approach was established that is likewise sensitive for AGO conformation.

To establish a specific and sensitive ELISA approach, we tested 16 CBA-positive neurological patients, 3 CBA-positive control diseases, 10 healthy controls and one commercial anti-AGO1 antibody (as a positive control) via ELISA using AGO1 protein in three conditions. Wells were coated using recombinant AGO1 diluted in:
(1) standard coating buffer (0.05 M carbonate/bicarbonate, pH 9.6 (Sigma-Aldrich, Saint-Louis, USA),
(2) in-house-adapted conformation-stabilizing coating buffer (30% glycerol in coating buffer), and
(3) in-house-adapted denaturizing coating buffer (0.8% SDS), in order to linearize AGO1.

For each of the three conditions a respective positivity threshold was defined based on the ΔODs of the 10 healthy controls under the respective conditions (horizontal bars=arithmetic mean plus 3 standard deviations).

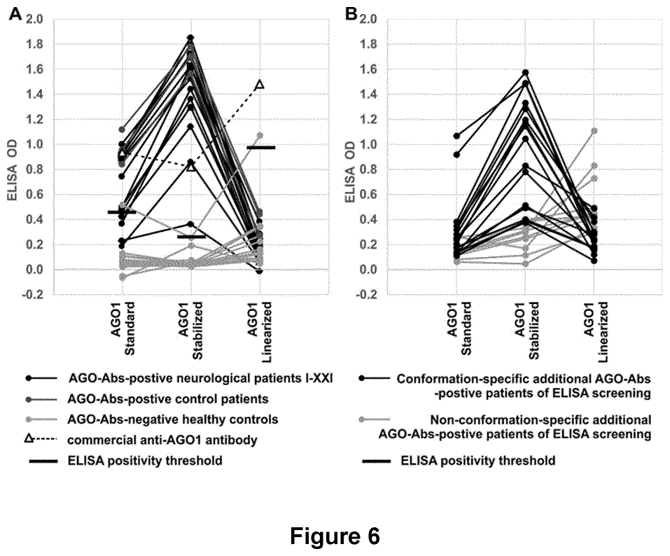

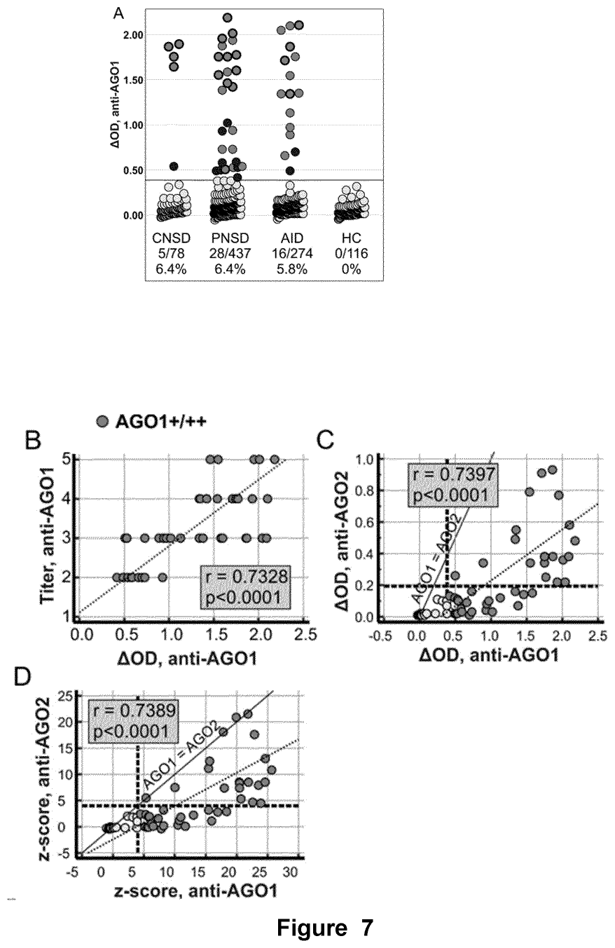

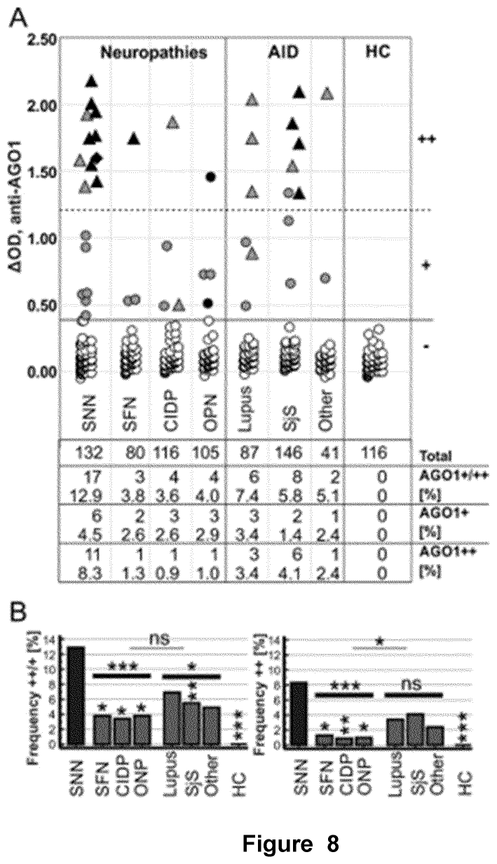

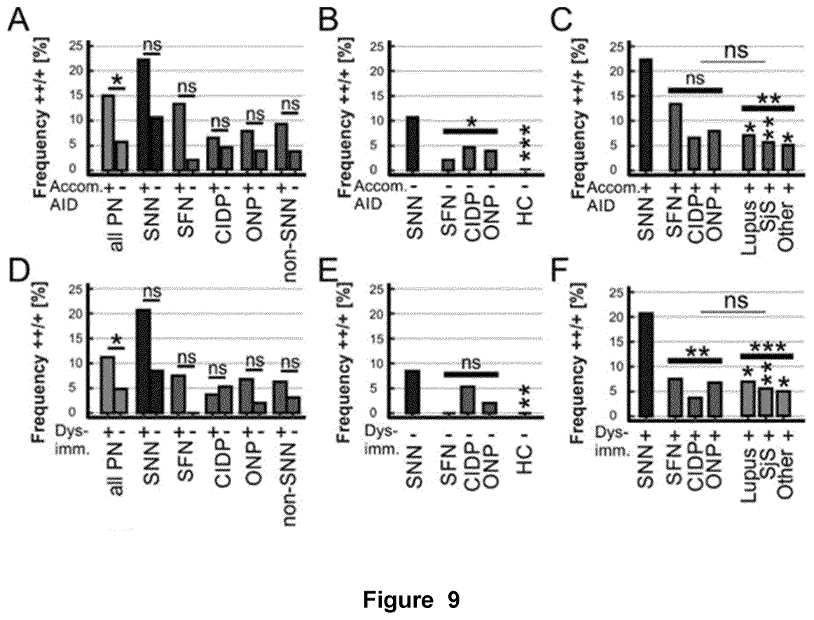

The positive control, commercial anti-AGO1 antibody resulted in strong signals in all three conditions (FIG. 6A), but the strongest signal was observed with the linearized AGO1.

Regarding the human sera, under the conformation-stabilizing conditions, all 19 (16+3; 100%) CBA-positive sera were confirmed via ELISA, while at the same time none (0%) of the 10 healthy controls was positive. In contrast, ELISA under standard conditions was positive in only 12/16 CBA-positive sera (75%), while 1/10 of the healthy controls was false-positive (10%).

These results suggest that ELISA in conformation-stabilizing conditions could be used to detect anti-AGO1 antibodies with a good sensitivity and specificity.

### Example 8. AGO1-Abs Detected by ELISA in Conformation-Stabilizing Show a Clear Association with a Subgroup of Patients with SNN

Using the conformation-stabilizing ELISA described in example 7, we first tested a new cohort of 823 sera, including 116 healthy controls to calibrate the test. The ODs of the healthy controls were used to define an adapted positivity threshold.

Using this new cohort, we identified 27 novel AGO Abs-positive patients (FIG. 6B).

All the 116 healthy controls were negative, but 5 (6.4%) of 78 subjects with CNS diseases, 28 (6.4%) of 437 subjects with different kind of neuropathies and 16 (5.8%) of 274 subjects with systemic autoimmune diseases (AID) had AGO1 Abs (FIG. 7A).

AGO1 Abs titers correlated with ELISA reactivity (FIG. 7B).

AGO2 Abs ELISA reactivity correlated with AGO1 Abs ELISA reactivity, but the sensitivity for AGO1 Abs was higher than for AGO2 Abs (FIG. 7C). Even when using the z-scores for which both AGO1 and AGO2 ELISA results have been normalized with their respective control cohort, most patient sera bind AGO1 more sensitively than AGO2 (FIG. 7D).

In a second cohort of patients, and as SNN was the most frequent disorder associated with AGO Abs (FIG. 8A, 8B), a specific test regarding the 132 patients with SNN according to the published criteria (Camdessanché et al. 2009) was performed, and compared to 116 with chronic inflammatory demyelinating polyneuropathies (CIDP), 80 with small-fiber neuropathies (SFN), and 105 with other neuropathies (ONP; mostly length dependent axonal sensory-motor neuropathies).

We also tested as control 87 patients with systemic lupus erythematosus (SLE), 146 with Sjögren Syndrome (SjS), and 41 with other autoimmune diseases (AID) including autoimmune hepatitis, primary biliary cirrhosis, systemic sclerosis, systemic vasculitis, myositis, rheumatoid arthritis, or juvenile arthritis.

We identified 44 have AGO1 Abs (FIG. 8A): 0/116 Healthy Controls, 17/132 SNN (12.9%), 3/80 (3.8%) SFN, 4/116 CIDP (3.6%), and 4/105 ONP (4.0%).

Out of the 274 patients with AID, 6/87 SLE (7.4%), 8/146 SjS (5.8%), 2/41 other AID (5.1%) had AGO1 Abs.

AGO1 Abs were more frequent with SNN as compared to non-SNN neuropathies (p=0.001, Fisher's exact test), AID (p=0.02), and HC (p<0.0001).

Among neuropathies, anti-AGO1 Abs were more frequent with SNN as compared to SFN (p=0.03), CIDP (p=0.01), or ONP (p=0.02).

Among neuropathies, AGO1 Abs were more frequent in patients with an associated AID (12/80, 15.0%) against 16/281, 5.7% in patients without associated AID (p=0.02).

Among all patients (neuropathies and AID) with AGO1 Abs, there was a good correlation between the antibody titer as measured by end-point dilution and the Z-score between AGO1 and AGO2 Abs (see FIG. 7D).

AGO1 Abs titers measured by end-point dilution ranged from 100-100,000 in the neuropathy cohorts and from 100-10,000 in the AID cohort. The frequency of patients with high titer of AGO1 Abs (>10,000) was higher with SNN (11/132 [8.3%]) as compared to non-SNN neuropathies (3/301 [1.0%], p=0.0002), healthy controls (0/116 [0%], p=0.001 and AID (10/276, 3.6%, p=0.044).

We then compared the clinical pattern of the SNN in patients with (17) and without (115) AGO1 Abs. There was no difference in term of age and sex, but patients with AGO1 Abs have a higher SNN score (median 12.2 [25-75%-intIQR: 11-12.7] vs. 11.0 [8.2-11], p=0.004), the face was more frequently affected (8/17 [47%] vs. 14/75 [19%], p=0.01), global areflexia was more frequent (13/17 [76%] vs. 29/75 [39%], p=0.01) and the m-RANKIN score was higher in patients with AGO1 Abs (Odds ratio (OR) 3.63, 1.74-7.57 95% CI, p=0.001). Regarding ENMG, the number of abolished SNAPs was higher in AGO1 Abs-positive SNN s (2.6±0.4 [72.1%] vs 1.8±0.2 [55.9%]; p=0.046).

These results show that AGO1 Abs characterize a group of patients with SNN presenting a more severe and widespread disorder that may be improved by immunomodulatory treatments.

### REFERENCES

U.S. Pat. No. 10,539,577
U.S. Pat. No. 9,766,253
U.S. Pat. No. 10,539,577
U.S. Pat. No. 7,314,721
EP 2952898
EP 3086120
US 2017/0343564
EP 3026434
EP 3101424
Antoine J-C, Boutahar N, Lassablière F, et al. Antifibroblast growth factor receptor 3 antibodies identify a subgroup of patients with sensory neuropathy. J Neurol Neurosurg Psychiatry. 2015; 86:1347-1355.
Bhanji R A, Eystathioy T, Chan E K L, Bloch D B, Fritzler M J. Clinical and serological features of patients with autoantibodies to GW/P bodies. Clin Immunol Orlando Fla. 2007; 125:247-256.
Camdessanché JP, Jousserand G, Ferraud K, Vial C, Petiot P, Honnorat J, Antoine J C. The pattern and diagnostic criteria of sensory neuronopathy: a case-control study. Brain. 2009 July; 132(Pt 7):1723-33. Casabona M G, Vandenbrouck Y, Attree I, Couté Y. Proteomic characterization of Pseudomonas aeruginosa PA01 inner membrane. Proteomics. 2013; 13:2419-2423.
Dalmau J, Geis C, Graus F. Autoantibodies to Synaptic Receptors and Neuronal Cell Surface Proteins in Autoimmune Diseases of the Central Nervous System. Physiol Rev. 2017; 97:839-887.
Graus F. Recommended diagnostic criteria for paraneoplastic neurological syndromes. J Neurol Neurosurg Psychiatry. 2004; 75:1135-1140.
Obermaier C, Griebel A, Westermeier R. Principles of Protein Labeling Techniques. Methods Mol Biol. 2021; 2261:549-562. Jakymiw A, Ikeda K, Fritzler M J, Reeves W H, Satoh M, Chan E K L. Autoimmune targeting of key components of RNA interference. Arthritis Res Ther. 2006; 8:R87.
Meister G, Landthaler M, Peters L, et al. Identification of novel argonaute-associated proteins. Curr Biol CB. 2005; 15:2149-2155.
Moritz C P, Tholance Y, Lassabliére F, Camdessanché J-P, Antoine J-C. Reducing the risk of misdiagnosis of indirect ELISA by normalizing serum-specific background noise: The example of detecting anti-FGFR3 autoantibodies. J Immunol Methods. 2019; 466:52-56.
Peters L, Meister G. Argonaute proteins: mediators of RNA silencing. Mol Cell. 2007; 26:611-623.
Querol L, Devaux J, Rojas-Garcia R, Illa I. Autoantibodies in chronic inflammatory neuropathies: diagnostic and therapeutic implications. Nat Rev Neurol. 2017; 13:533-547.
Satoh M, Langdon J J, Chou C H, et al. Characterization of the Su antigen, a macromolecular complex of 100/102 and 200-kDa proteins recognized by autoantibodies in systemic rheumatic diseases. Clin Immunol Immunopathol. 1994; 73:132-141.
Satoh M, Chan J Y F, Ceribelli A, Vazquez del-Mercado M, Chan E K L. Autoantibodies to Argonaute 2 (Su antigen). Adv Exp Med Biol. 2013; 768:45-59.
Tholance Y, Moritz C P, Rosier C, et al. Clinical characterisation of sensory neuropathy with anti-FGFR3 autoantibodies J Neurol Neurosurg Psychiatry epub ahead of print. doi:10.1136/jnnp-2019-321849.

1-13. (canceled)

14. Process for identifying patients affected by an autoimmune neurological disease, comprising a step of detection of at least one type of anti-Argonaute autoantibodies (AGO-Abs) in a biological sample of an individual susceptible to be affected by said disease, wherein positive detection of said at least one type of AGO-Abs means that said individual is affected by said autoimmune neurological disease.

15. Process according to claim 14, wherein said at least one type of AGO-Abs reacts with only one single AGO protein, or reacts with any combination of two AGO proteins, or reacts with any combination of three AGO proteins, or reacts with the four proteins AGO1, AGO2, AGO3 and AGO4.

16. Process according to claim 14, wherein the biological sample is selected among the group consisting of: cerebrospinal fluid, serum, plasma, whole blood, urine, lymph, saliva, sputum, seminal fluid and tears.

17. Process according to claim 14, wherein the step of detection of AGO-Abs is performed by cell-based assay (CBA) with a panel of cells, each cell expressing separately AGO1, AGO2, AGO3 or AGO4 protein, or at least one of their fragments.

18. Process according to claim 14, wherein the step of detection of AGO-Abs is performed by an immunoassay.

19. Process according to claim 18, wherein the immunoassay is chosen among the group consisting of: ELISA, immunoblot, radio-immunoassay, chemiluminescent immunoassay, electro-chemiluminescence immunoassay, and immunofluorescence assay.

20. Process according to claim 19, wherein the immunoblot is chosen among the group consisting of: dot blot, western blot, and line assay.

21. Process according to claim 18, wherein the immunoassay is ELISA.

22. Process according to claim 21 wherein the ELISA is an ELISA under conformation-stabilizing conditions.

23. Process according to claim 14, wherein the step of detection of at least one type of AGO-Abs is coupled with a step a quantification of said AGO-Abs.

24. Process according to claim 14, wherein the autoimmune neurological disease affecting the identified patients is chosen among the group consisting of: autoimmune encephalitis, paraneoplastic neurological syndromes and inflammatory peripheral neuropathies.

25. Process according to claim 24 wherein the autoimmune neurological disease is chosen among the group consisting of: sensory neuronopathy, limbic encephalitis, cerebellar syndrome, and other inflammatory peripheral neuropathies.

26. Process according to claim 25 wherein the other inflammatory peripheral neuropathies are chosen among the group consisting of: small fiber neuropathy, chronic inflammatory demyelinating polyneuropathy, rhombencephalitis, and opsoclonus-myoclonus.

27. Process according to claim 25, wherein the autoimmune neurological disease affecting the identified patients is an autoimmune sensory neuronopathy.

28. Process of classification of patients, comprising:
a. identifying among a group of patients affected with neurological symptoms a subgroup of patients affected with an autoimmune neurological disease, wherein the step of identification is made by the process according to claim 14, and
b. classifying said patients.

29. Biomarker specific of the autoimmune nature of a neurological disease, consisting of Argonaute autoantibodies (AGO-Abs).

30. Biomarker specific of the autoimmune nature of a neurological disease according to claim 29, consisting of AGO-Absdirected against at least one of the following proteins: AGO1, AGO2, AGO3, AGO4, and combinations thereof.

31. Kit for the implementation of the process according to claim 14, comprising means for the detection and/or quantification of at least one type of AGO-Abs in a biological sample of an individual.

32. Kit according to claim 31, comprising:
a. at least one antigen or antigen-expressing cell or nucleic acid encoding an antigen, chosen among the group consisting of:
i. Cells overexpressing selectively AGO1, AGO2, AGO3, or AGO4 proteins, or a variant thereof, or any combination thereof;
ii. AGO1, AGO2, AGO3 or AGO4 protein, or a variant thereof, or any combination thereof;
iii. A fragment or peptide issued from AGO1, AGO2, AGO3, or AGO4 protein, or a variant thereof, or any combination thereof;
iv. An expression vector carrying at least one nucleic acid encoding AGO1, AGO2, AGO3, or AGO4 protein, or a variant thereof, or any fragment or peptide issued from AGO1, AGO2, AGO3, or AGO4 protein, or a variant thereof, or any combination thereof;
b. Anti-human immunoglobulin antibody coupled to a probe; and
c. Reactant(s) useful for the in vitro step of detection and/or quantification.

33. The kit according to claim 32, comprising a fragment or peptide issued from AGO1, AGO2, AGO3, or AGO4 protein, or a variant thereof, coated on a solid carrier.

34. The kit according to claim 33, whether said solid carrier is selected from the group consisting in: a glass slide, a biochip, a microtiter plate, a lateral flow device, a test strip, a membrane, a chromatography column and a bead.

35. A process of treatment of an autoimmune neurological disease in patients in need thereof, comprising the following steps:
a) classifying patients affected with neurological diseases according to the process of classification according to claim 14, and
b) administering an appropriate immunomodulatory compound to a subgroup of patients identified as being affected with an autoimmune neurological disease.


SEQUENCE LISTING (8 sequences)

SEQ ID NO: 1 - DNA, 7884 bp, Artificial Sequence
  Plasmid VP5-HA-AGO1
aattcagtggatccactagtaacggccgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatcgccgccgccatggactacaaggacgacgatgacaagtacccttatgacgtgcccgattacgctagcggccgcatggaagcgggaccctcgggagcagctgcgggcgcttacctgccccccctgcagcaggtgttccaggcacctcgccggcctggcattggcactgtggggaaaccaatcaagctcctggccaattactttgaggtggacatccctaagatcgacgtgtaccactacgaggtggacatcaagccggataagtgtccccgtagagtcaaccgggaagtggtggaatacatggtccagcatttcaagcctcagatctttggtgatcgcaagcctgtgtatgatggaaagaagaacatttacactgtcacagcactgcccattggcaacgaacgggtcgactttgaggtgacaatccctggggaagggaaggatcgaatctttaaggtctccatcaagtggctagccattgtgagctggcgaatgctgcatgaggccctggtcagcggccagatccctgttcccttggagtctgtgcaagccctggatgtggccatgaggcacctggcatccatgaggtacacccctgtgggccgctccttcttctcaccgcctgagggctactaccacccgctggggggtgggcgcgaggtctggttcggctttcaccagtctgtgcgccctgccatgtggaagatgatgctcaacattgatgtctcagccactgccttttataaggcacagccagtgattgagttcatgtgtgaggtgctggacatcaggaacatagatgagcagcccaagcccctcacggactctcagcgcgttcgcttcaccaaggagatcaagggcctgaaggtggaagtcacccactgtggacagatgaagaggaagtaccgcgtgtgtaatgttacccgtcgccctgctagccatcagacattccccttacagctggagagtggacagactgtggagtgcacagtggcacagtatttcaagcagaaatataaccttcagctcaagtatccccatctgccctgcctacaagttggccaggaacaaaagcatacctaccttcccctagaggtctgtaacattgtggctgggcagcgctgtattaaaaagctgaccgacaaccagacctcgaccatgataaaggccacagctagatccgctccagacagacaggaggagatcagtcgcctgatgaagaatgccagctacaacttagatccctacatccaggaatttgggatcaaagtgaaggatgacatgacggaggtgacagggcgagtgctgccggcgcccatcttgcagtacggcggccggaaccgggccattgccacacccaatcagggtgtctgggacatgcgggggaaacagttctacaatgggattgagatcaaagtctgggccatcgcctgcttcgcaccccaaaaacagtgtcgagaagaggtgctcaagaacttcacagaccagctgcggaagatttccaaggatgcggggatgcctatccagggtcaaccttgtttctgcaaatatgcacagggggcagacagcgtggagcctatgttccggcatctcaagaacacctactcagggctgcagctcattattgtcatcctgccagggaagacgccggtgtatgctgaggtgaaacgtgtcggagatacactcttgggaatggctacgcagtgtgtgcaggtgaagaacgtggtcaagacctcacctcagactctgtccaacctctgcctcaagatcaatgtcaaacttggtggcattaacaacatcctagtcccacaccagcgctctgccgtttttcaacagccagtgatattcctgggagcagatgttacacaccccccagcaggggatgggaaaaaaccttctatcacagcagtggtaggcagtatggatgcccaccccagccgatactgtgctactgtgcgggtacagcgaccacggcaagagatcattgaagacttgtcctacatggtgcgtgagctcctcatccaattctacaagtccacccgtttcaagcctacccgcatcatcttctaccgagatggggtgcctgaaggccagctaccccagatactccactatgagctactggccattcgtgatgcctgcatcaaactggaaaaggactaccagcctgggatcacttatattgtggtgcagaaacgccatcacacccgccttttctgtgctgacaagaatgagcgaattgggaagagtggtaacatcccagctgggaccacagtggacaccaacatcacccacccatttgagtttgacttctatctgtgcagccacgcaggcatccagggcaccagccgaccatcccattactatgttctttgggatgacaaccgtttcacagcagatgagctccagatcctgacgtaccagctgtgccacacttacgtacgatgcacacgctctgtctctatcccagcacctgcctactatgcccgcctggtggctttccgggcacgataccacctggtggacaaggagcatgacagtggagaggggagccacatatcggggcagagcaatgggcgggacccccaggccctggccaaagccgtgcaggttcaccaggatactctgcgcaccatgtacttcgcttgag

SEQ ID NO: 2 - DNA, 7890 bp, Artificial Sequence
  Plasmid VP5-HA-AGO2
aattcagtggatccactagtaacggccgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatcgccgccgccatggactacaaggacgacgatgacaagtacccttatgacgtgcccgattacgctagcggccgcatgtactcgggagccggccccgcacttgcacctcctgcgccgccgccccccatccaaggatatgccttcaagcctccacctagacccgactttgggacctccgggagaacaatcaaattacaggccaatttcttcgaaatggacatccccaaaattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgtgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcgactgctaggtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgccggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagacgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgacggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttgag

SEQ ID NO: 3 - DNA, 7893 bp, Artificial Sequence
  Plasmid VP5-HA-AGO3
aattcagtggatccactagtaacggccgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatcgccgccgccatggactacaaggacgacgatgacaagtacccttatgacgtgcccgattacgctagcggccgcatggaaatcggctccgcaggacccgctggggcccagcccctactcatggtgcccagaagacctggctatggcaccatgggcaaacccattaaactgctggctaactgttttcaagttgaaatcccaaagattgatgtctacctctatgaggtagatattaaaccagacaagtgtcctaggagagtgaacagggaggtggttgactcaatggttcagcattttaaagtaactatatttggagaccgtagaccagtttatgatggaaaaagaagtctttacaccgccaatccacttcctgtggcaactacaggggtagatttagacgttactttacctggggaaggtggaaaagatcgacctttcaaggtgtcaatcaaatttgtctctcgggtgagttggcacctactgcatgaagtactgacaggacggaccttgcctgagccactggaattagacaagccaatcagcactaaccctgtccatgccgttgatgtggtgctacgacatctgccctccatgaaatacacacctgtggggcgttcatttttctccgctccagaaggatatgaccaccctctgggagggggcagggaagtgtggtttggattccatcagtctgttcggcctgccatgtggaaaatgatgcttaatatcgatgtttctgccactgccttctacaaagcacaacctgtaattcagttcatgtgtgaagttcttgatattcataatattgatgagcaaccaagacctctgactgattctcatcgggtaaaattcaccaaagagataaaaggtttgaaggttgaagtgactcattgtggaacaatgagacggaaataccgtgtttgtaatgtaacaaggaggcctgccagtcatcaaacctttcctttacagttagaaaacggccaaactgtggagagaacagtagcgcagtatttcagagaaaagtatactcttcagctgaagtacccgcaccttccctgtctgcaagtcgggcaggaacagaaacacacctacctgccactagaagtctgtaatattgtggcagggcaacgatgtatcaagaagctaacagacaatcagacttccactatgatcaaggcaacagcaagatctgcaccagatagacaagaggaaattagcagattggtaagaagtgcaaattatgaaacagatccatttgttcaggagtttcaatttaaagttcgggatgaaatggctcatgtaactggacgcgtacttccagcacctatgctccagtatggaggacggaatcggacagtagcaacaccgagccatggagtatgggacatgcgagggaaacaattccacacaggagttgaaatcaaaatgtgggctatcgcttgttttgccacacagaggcagtgcagagaagaaatattgaagggtttcacagaccagctgcgtaagatttctaaggatgcagggatgcccatccagggccagccatgcttctgcaaatatgcacagggggcagacagcgtagagcccatgttccggcatctcaagaacacatattctggcctacagcttattatcgtcatcctgccggggaagacaccagtgtatgcggaagtgaaacgtgtaggagacacacttttgggtatggctacacaatgtgttcaagtcaagaatgtaataaaaacatctcctcaaactctgtcaaacttgtgcctaaagataaatgttaaactcggagggatcaataatattcttgtacctcatcaaagaccttctgtgttccagcaaccagtgatctttttgggagccgatgtcactcatccacctgctggtgatggaaagaagccttctattgctgctgttgtaggtagtatggatgcacacccaagcagatactgtgccacagtaagagttcagagaccccgacaggagatcatccaggacttggcctccatggtccgggaacttcttattcaattttataagtcaactcggttcaagcctactcgtatcatcttttatcgggatggtgtttcagaggggcagtttaggcaggtattatattatgaactactagcaattcgagaagcctgcatcagtttggagaaagactatcaacctggaataacctacattgtagttcagaagagacatcacactcgattattttgtgctgataggacagaaagggttggaagaagtggcaatatcccagctggaacaacagttgatacagacattacacacccatatgagttcgatttttacctctgtagccatgctggaatacagggtaccagtcgtccttcacactatcatgttttatgggatgataactgctttactgcagatgaacttcagctgctaacttaccagctctgccacacttacgtacgctgtacacgatctgtttctatacctgcaccagcgtattatgctcacctggtagcatttagagccagatatcatcttgtggacaaagaacatgacagtgctgaaggaagtcacgtttcaggacaaagcaatgggcgagatccacaagctcttgccaaggctgtacagattcaccaagataccttacgcacaatgtacttcgcttaag

SEQ ID NO: 4 - DNA, 7833 bp, Artificial Sequence
  Plasmid VP5-HA-AGO4
aattcagtggatccactagtaacggccgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatcgccgccgccatggaggcgctgggacccggacctccggctagcctgtttcagccacctcgtcgtcctggccttggaactgttggaaaaccaattcgactgttagccaatcattttcaggttcagattcctaaaatagatgtgtatcactatgatgtggatattaagcctgaaaaacggcctcgtagagtcaacagggaggtagtagatacaatggtgcggcacttcaagatgcaaatatttggtgatcggcagcctgggtatgatggcaaaagaaacatgtacacagcacatccactaccaattggacgggatagggttgatatggaggtgactcttccaggcgagggtaaagaccaaacatttaaagtgtctgttcagtgggtgtcagttgtgagccttcagttgcttttagaagctttggctgggcacttgaatgaagtcccagatgactcagtacaagcacttgatgttatcacaagacaccttccctccatgaggtacaccccagtgggccgttcctttttctcacccccggaaggttactaccaccctctgggagggggcagggaggtctggtttggttttcatcagtctgtgagacctgccatgtggaatatgatgctcaacattgatgtatctgcaactgctttctaccgggctcagcctatcattgagttcatgtgtgaggttttagacattcagaacatcaatgaacagaccaaacctctaacagactcccagcgtgtcaaatttaccaaagaaatcagaggtctcaaagttgaggtgacccactgtggacagatgaaacgaaaataccgagtttgtaatgtgactagacggccagccagtcatcaaacttttcctttgcagctagaaaacggtcaagctatggaatgtacagtagctcaatattttaagcaaaagtatagtctgcaactgaaatacccccatcttccctgtctccaagtgggacaagaacaaaagcatacatacttgccactcgaggtctgtaatatagtggcaggacagcgatgtatcaagaagctcacagacaatcagacttccacaatgatcaaagctacagcaagatctgctcctgacagacaggaagagatcagtagactggtgaagagcaacagtatggtgggtggacctgatccataccttaaagaatttggtattgttgtccacaatgaaatgacagagctcacaggcagggtacttccagcaccaatgctgcaatatggaggccggaataaaacagtagccacacccaaccagggtgtctgggacatgcgaggaaagcagttttatgctggcattgaaattaaagtttgggcagttgcttgttttgcacctcagaaacaatgtagggaagatttactaaagagtttcactgaccagctgcgtaaaatctctaaggatgcaggaatgcccatccagggtcagccatgtttctgcaagtatgcacaaggtgcagacagtgtggagcctatgtttaaacatctgaaaatgacttatgtgggcctacagctaatagtggttatcctgcctggaaagacaccagtatatgcggaggtgaaacgtgttggagatacccttctaggtatggccacacagtgtgtccaggtaaaaaatgtagtgaagacctcacctcaaaccctttccaatctttgcctgaagataaatgcaaaacttggaggaattaacaatgtgcttgtgcctcatcaaaggccctcggtgttccagcagcctgtcatcttcctgggagcggatgtcacacaccccccagcaggggatgggaagaaaccttccattgctgctgtggttggcagtatggatggccaccccagccggtactgtgccaccgttcgggtgcagacttcccggcaggagatctcccaagagctcctctacagtcaagaggtcatccaggacctgactaacatggttcgagagctgctgattcagttctacaaatccacacgcttcaaacccactcggatcatctattaccgtggaggggtatctgagggacaaatgaaacaggtagcttggccagaactaatagcaattcgaaaggcatgtattagcttggaagaagattaccggccaggaataacttatattgtggtgcaaaaaagacatcacacacgactcttctgtgcagataaaacagaaagggtagggaaaagtggcaatgtaccagcaggcactacagtggatagtaccatcacacatccatctgagtttgacttttacctctgtagtcatgcaggaattcagggaaccagccgtccctcacattaccaggtcttgtgggatgacaactgcttcactgcagatgaactccagctactgacttaccagctgtgtcacacctatgtgaggtgcactcgctcagtctctattccagcccctgcatattatgcccggcttgtagcatttagggcaaggtatcatctggtggataaagatcatgacagtgcggaaggcagtcatgtgtcaggacagagcaacggccgggatcctcaggccttggctaaggctgtgcaaatccaccatgatacccagcacacgatgtattttgcctgag

SEQ ID NO: 5 - DNA, 2574 bp, Homo sapiens
atggaagcgggaccctcgggagcagctgcgggcgcttacctgccccccctgcagcaggtgttccaggcacctcgccggcctggcattggcactgtggggaaaccaatcaagctcctggccaattactttgaggtggacatccctaagatcgacgtgtaccactacgaggtggacatcaagccggataagtgtccccgtagagtcaaccgggaagtggtggaatacatggtccagcatttcaagcctcagatctttggtgatcgcaagcctgtgtatgatggaaagaagaacatttacactgtcacagcactgcccattggcaacgaacgggtcgactttgaggtgacaatccctggggaagggaaggatcgaatctttaaggtctccatcaagtggctagccattgtgagctggcgaatgctgcatgaggccctggtcagcggccagatccctgttcccttggagtctgtgcaagccctggatgtggccatgaggcacctggcatccatgaggtacacccctgtgggccgctccttcttctcaccgcctgagggctactaccacccgctggggggtgggcgcgaggtctggttcggctttcaccagtctgtgcgccctgccatgtggaagatgatgctcaacattgatgtctcagccactgccttttataaggcacagccagtgattgagttcatgtgtgaggtgctggacatcaggaacatagatgagcagcccaagcccctcacggactctcagcgcgttcgcttcaccaaggagatcaagggcctgaaggtggaagtcacccactgtggacagatgaagaggaagtaccgcgtgtgtaatgttacccgtcgccctgctagccatcagacattccccttacagctggagagtggacagactgtggagtgcacagtggcacagtatttcaagcagaaatataaccttcagctcaagtatccccatctgccctgcctacaagttggccaggaacaaaagcatacctaccttcccctagaggtctgtaacattgtggctgggcagcgctgtattaaaaagctgaccgacaaccagacctcgaccatgataaaggccacagctagatccgctccagacagacaggaggagatcagtcgcctgatgaagaatgccagctacaacttagatccctacatccaggaatttgggatcaaagtgaaggatgacatgacggaggtgacagggcgagtgctgccggcgcccatcttgcagtacggcggccggaaccgggccattgccacacccaatcagggtgtctgggacatgcgggggaaacagttctacaatgggattgagatcaaagtctgggccatcgcctgcttcgcaccccaaaaacagtgtcgagaagaggtgctcaagaacttcacagaccagctgcggaagatttccaaggatgcggggatgcctatccagggtcaaccttgtttctgcaaatatgcacagggggcagacagcgtggagcctatgttccggcatctcaagaacacctactcagggctgcagctcattattgtcatcctgccagggaagacgccggtgtatgctgaggtgaaacgtgtcggagatacactcttgggaatggctacgcagtgtgtgcaggtgaagaacgtggtcaagacctcacctcagactctgtccaacctctgcctcaagatcaatgtcaaacttggtggcattaacaacatcctagtcccacaccagcgctctgccgtttttcaacagccagtgatattcctgggagcagatgttacacaccccccagcaggggatgggaaaaaaccttctatcacagcagtggtaggcagtatggatgcccaccccagccgatactgtgctactgtgcgggtacagcgaccacggcaagagatcattgaagacttgtcctacatggtgcgtgagctcctcatccaattctacaagtccacccgtttcaagcctacccgcatcatcttctaccgagatggggtgcctgaaggccagctaccccagatactccactatgagctactggccattcgtgatgcctgcatcaaactggaaaaggactaccagcctgggatcacttatattgtggtgcagaaacgccatcacacccgccttttctgtgctgacaagaatgagcgaattgggaagagtggtaacatcccagctgggaccacagtggacaccaacatcacccacccatttgagtttgacttctatctgtgcagccacgcaggcatccagggcaccagccgaccatcccattactatgttctttgggatgacaaccgtttcacagcagatgagctccagatcctgacgtaccagctgtgccacacttacgtacgatgcacacgctctgtctctatcccagcacctgcctactatgcccgcctggtggctttccgggcacgataccacctggtggacaaggagcatgacagtggagaggggagccacatatcggggcagagcaatgggcgggacccccaggccctggccaaagccgtgcaggttcaccaggatactctgcgcaccatgtacttcgcttga

SEQ ID NO: 6 - DNA, 2580 bp, Homo sapiens
atgtactcgggagccggccccgcacttgcacctcctgcgccgccgccccccatccaaggatatgccttcaagcctccacctagacccgactttgggacctccgggagaacaatcaaattacaggccaatttcttcgaaatggacatccccaaaattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgtgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcgactgctaggtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgccggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagacgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgacggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttga

SEQ ID NO: 7 - DNA, 2583 bp, Homo sapiens
atggaaatcggctccgcaggacccgctggggcccagcccctactcatggtgcccagaagacctggctatggcaccatgggcaaacccattaaactgctggctaactgttttcaagttgaaatcccaaagattgatgtctacctctatgaggtagatattaaaccagacaagtgtcctaggagagtgaacagggaggtggttgactcaatggttcagcattttaaagtaactatatttggagaccgtagaccagtttatgatggaaaaagaagtctttacaccgccaatccacttcctgtggcaactacaggggtagatttagacgttactttacctggggaaggtggaaaagatcgacctttcaaggtgtcaatcaaatttgtctctcgggtgagttggcacctactgcatgaagtactgacaggacggaccttgcctgagccactggaattagacaagccaatcagcactaaccctgtccatgccgttgatgtggtgctacgacatctgccctccatgaaatacacacctgtggggcgttcatttttctccgctccagaaggatatgaccaccctctgggagggggcagggaagtgtggtttggattccatcagtctgttcggcctgccatgtggaaaatgatgcttaatatcgatgtttctgccactgccttctacaaagcacaacctgtaattcagttcatgtgtgaagttcttgatattcataatattgatgagcaaccaagacctctgactgattctcatcgggtaaaattcaccaaagagataaaaggtttgaaggttgaagtgactcattgtggaacaatgagacggaaataccgtgtttgtaatgtaacaaggaggcctgccagtcatcaaacctttcctttacagttagaaaacggccaaactgtggagagaacagtagcgcagtatttcagagaaaagtatactcttcagctgaagtacccgcaccttccctgtctgcaagtcgggcaggaacagaaacacacctacctgccactagaagtctgtaatattgtggcagggcaacgatgtatcaagaagctaacagacaatcagacttccactatgatcaaggcaacagcaagatctgcaccagatagacaagaggaaattagcagattggtaagaagtgcaaattatgaaacagatccatttgttcaggagtttcaatttaaagttcgggatgaaatggctcatgtaactggacgcgtacttccagcacctatgctccagtatggaggacggaatcggacagtagcaacaccgagccatggagtatgggacatgcgagggaaacaattccacacaggagttgaaatcaaaatgtgggctatcgcttgttttgccacacagaggcagtgcagagaagaaatattgaagggtttcacagaccagctgcgtaagatttctaaggatgcagggatgcccatccagggccagccatgcttctgcaaatatgcacagggggcagacagcgtagagcccatgttccggcatctcaagaacacatattctggcctacagcttattatcgtcatcctgccggggaagacaccagtgtatgcggaagtgaaacgtgtaggagacacacttttgggtatggctacacaatgtgttcaagtcaagaatgtaataaaaacatctcctcaaactctgtcaaacttgtgcctaaagataaatgttaaactcggagggatcaataatattcttgtacctcatcaaagaccttctgtgttccagcaaccagtgatctttttgggagccgatgtcactcatccacctgctggtgatggaaagaagccttctattgctgctgttgtaggtagtatggatgcacacccaagcagatactgtgccacagtaagagttcagagaccccgacaggagatcatccaggacttggcctccatggtccgggaacttcttattcaattttataagtcaactcggttcaagcctactcgtatcatcttttatcgggatggtgtttcagaggggcagtttaggcaggtattatattatgaactactagcaattcgagaagcctgcatcagtttggagaaagactatcaacctggaataacctacattgtagttcagaagagacatcacactcgattattttgtgctgataggacagaaagggttggaagaagtggcaatatcccagctggaacaacagttgatacagacattacacacccatatgagttcgatttttacctctgtagccatgctggaatacagggtaccagtcgtccttcacactatcatgttttatgggatgataactgctttactgcagatgaacttcagctgctaacttaccagctctgccacacttacgtacgctgtacacgatctgtttctatacctgcaccagcgtattatgctcacctggtagcatttagagccagatatcatcttgtggacaaagaacatgacagtgctgaaggaagtcacgtttcaggacaaagcaatgggcgagatccacaagctcttgccaaggctgtacagattcaccaagataccttacgcacaatgtacttcgcttaa

SEQ ID NO: 8 - DNA, 2586 bp, Homo spaiens
atggaggcgctgggacccggacctccggctagcctgtttcagccacctcgtcgtcctggccttggaactgttggaaaaccaattcgactgttagccaatcattttcaggttcagattcctaaaatagatgtgtatcactatgatgtggatattaagcctgaaaaacggcctcgtagagtcaacagggaggtagtagatacaatggtgcggcacttcaagatgcaaatatttggtgatcggcagcctgggtatgatggcaaaagaaacatgtacacagcacatccactaccaattggacgggatagggttgatatggaggtgactcttccaggcgagggtaaagaccaaacatttaaagtgtctgttcagtgggtgtcagttgtgagccttcagttgcttttagaagctttggctgggcacttgaatgaagtcccagatgactcagtacaagcacttgatgttatcacaagacaccttccctccatgaggtacaccccagtgggccgttcctttttctcacccccggaaggttactaccaccctctgggagggggcagggaggtctggtttggttttcatcagtctgtgagacctgccatgtggaatatgatgctcaacattgatgtatctgcaactgctttctaccgggctcagcctatcattgagttcatgtgtgaggttttagacattcagaacatcaatgaacagaccaaacctctaacagactcccagcgtgtcaaatttaccaaagaaatcagaggtctcaaagttgaggtgacccactgtggacagatgaaacgaaaataccgagtttgtaatgtgactagacggccagccagtcatcaaacttttcctttgcagctagaaaacggtcaagctatggaatgtacagtagctcaatattttaagcaaaagtatagtctgcaactgaaatacccccatcttccctgtctccaagtgggacaagaacaaaagcatacatacttgccactcgaggtctgtaatatagtggcaggacagcgatgtatcaagaagctcacagacaatcagacttccacaatgatcaaagctacagcaagatctgctcctgacagacaggaagagatcagtagactggtgaagagcaacagtatggtgggtggacctgatccataccttaaagaatttggtattgttgtccacaatgaaatgacagagctcacaggcagggtacttccagcaccaatgctgcaatatggaggccggaataaaacagtagccacacccaaccagggtgtctgggacatgcgaggaaagcagttttatgctggcattgaaattaaagtttgggcagttgcttgttttgcacctcagaaacaatgtagggaagatttactaaagagtttcactgaccagctgcgtaaaatctctaaggatgcaggaatgcccatccagggtcagccatgtttctgcaagtatgcacaaggtgcagacagtgtggagcctatgtttaaacatctgaaaatgacttatgtgggcctacagctaatagtggttatcctgcctggaaagacaccagtatatgcggaggtgaaacgtgttggagatacccttctaggtatggccacacagtgtgtccaggtaaaaaatgtagtgaagacctcacctcaaaccctttccaatctttgcctgaagataaatgcaaaacttggaggaattaacaatgtgcttgtgcctcatcaaaggccctcggtgttccagcagcctgtcatcttcctgggagcggatgtcacacaccccccagcaggggatgggaagaaaccttccattgctgctgtggttggcagtatggatggccaccccagccggtactgtgccaccgttcgggtgcagacttcccggcaggagatctcccaagagctcctctacagtcaagaggtcatccaggacctgactaacatggttcgagagctgctgattcagttctacaaatccacacgcttcaaacccactcggatcatctattaccgtggaggggtatctgagggacaaatgaaacaggtagcttggccagaactaatagcaattcgaaaggcatgtattagcttggaagaagattaccggccaggaataacttatattgtggtgcaaaaaagacatcacacacgactcttctgtgcagataaaacagaaagggtagggaaaagtggcaatgtaccagcaggcactacagtggatagtaccatcacacatccatctgagtttgacttttacctctgtagtcatgcaggaattcagggaaccagccgtccctcacattaccaggtcttgtgggatgacaactgcttcactgcagatgaactccagctactgacttaccagctgtgtcacacctatgtgaggtgcactcgctcagtctctattccagcccctgcatattatgcccggcttgtagcatttagggcaaggtatcatctggtggataaagatcatgacagtgcggaaggcagtcatgtgtcaggacagagcaacggccgggatcctcaggccttggctaaggctgtgcaaatccaccatgatacccagcacacgatgtattttgcctga